(19) United States
(12) Patent Application Publication
(10) Pub. No.: US 2024/0382248 A1
(43) Pub. Date: Nov. 21, 2024

(54) CARDIAC ABLATION CATHETERS WITH SEGMENTED ENERGY DELIVERY ELEMENTS AND/OR ENERGY DELIVERY ELEMENTS HAVING ADJUSTABLE APERTURES

(71) Applicant: Focused Therapeutics, Inc., Salt Lake City, UT (US)

(72) Inventors: Paul Melman, Newton, MA (US); Meir M. Brosh, Newton Center, MA (US); Yonathan F. Melman, Salt Lake City, UT (US); James D. Isaacson, Salt Lake City, UT (US)

(21) Appl. No.: 18/410,935

(22) Filed: Jan. 11, 2024

Related U.S. Application Data
(60) Provisional application No. 63/467,571, filed on May 18, 2023.
(60) Provisional application No. 63/578,219, filed on Aug. 23, 2023.

(51) Int. Cl. A61B 18/14

(52) U.S. Cl. CPC A61B 18/1492; A61B 2017/00738; A61B 2018/00077; A61B 2018/00083; A61B 2018/00577; A61B 2018/1467

(57) ABSTRACT

Cardiac ablation catheters, including cardiac ablation catheters with segmented energy delivery elements and/or energy delivery elements having adjustable apertures, are described herein. In one embodiment, an ablation catheter includes (i) a shaft having a proximal end and a distal end opposite the proximal end, and (ii) an ablation electrode at the distal end of the shaft. The ablation electrode can include a first conductive segment and a second conductive segment different from the first conductive segment. The first conductive segment and the second conductive segment can be arranged in a stack along a common axis, and the first conductive segment and the second conductive segment can be independently energizable. In some embodiments, the first conductive segment and the second conductive segment can be arranged in the stack such that the ablation electrode has a partial dome shape with an adjustable aperture or oculus.

### CROSS-REFERENCE TO RELATED APPLICATIONS

The present application claims the benefit of U.S. Provisional Patent Application No. 63/467,571, filed May 18, 2023, and U.S. Provisional Patent Application No. 63/578,219, filed Aug. 23, 2023, both of which are incorporated herein by reference in their entireties.

### TECHNICAL FIELD

The present disclosure relates generally to cardiac ablation catheters. For example, several embodiments of the present technology relate to cardiac ablation catheters with segmented energy delivery elements and/or energy delivery elements having adjustable apertures, and associated systems, devices, and methods.

### BACKGROUND

Cardiac ablation is a procedure to treat cardiac arrhythmias (e.g., abnormal or irregular heart rhythms such as flutter, fibrillation, and/or tachycardia in an atria or ventricle of a heart). More specifically, cardiac ablation uses energy (e.g., radiofrequency, pulsed field ablation, cryoablation) to treat cardiac tissue to thereby block irregular electrical signals in the heart and restore a typical heartbeat. During cardiac ablation, energy is commonly delivered to cardiac tissue via a minimally invasive ablation catheter that has been introduced into a patient's heart via his/her veins or arteries. In this scenario, the catheter can be used to form one or more discrete points (e.g., discrete lesions) on the wall of the patient's heart by applying energy (e.g., electrical energy) to the wall. The applied energy damages tissue at the treatment site(s), terminating the tissue's electrical activity. In turn, abnormal electrical signals can be prevented from propagating through the treated tissue, thereby preventing arrhythmias.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. The drawings should not be taken to limit the disclosure to the specific embodiments shown, but are provided for explanation and understanding.

FIG. 1 is a partially schematic diagram of a catheter ablation system configured in accordance with various embodiments of the present technology.

FIG. 2 is a partially schematic side perspective view of a segmented ablation electrode configured in accordance with various embodiments of the present technology.

FIG. 3 is a partially schematic side view of the ablation electrode of FIG. 2 and of a line plot illustrating electric field strength over tissue depth in accordance with various embodiments of the present technology.

FIG. 4A is a partially schematic, partially transparent side view of a catheter tip section configured in accordance with various embodiments of the present technology.

FIG. 4B is a partially schematic, partially transparent end view of the catheter tip section of FIG. 4A.

FIG. 5A is a partially schematic, partially transparent side view of another catheter tip section configured in accordance with various embodiments of the present technology.

FIG. 5B is a partially schematic, partially transparent end view of the catheter tip section of FIG. 5A.

FIG. 6 is a partially schematic side view of another segmented ablation electrode configured in accordance with various embodiments of the present technology.

FIGS. 7A and 7B are partially schematic, transparent side views of another tip section configured in accordance with various embodiments of the present technology.

FIG. 8A is a partially schematic, transparent side view of another tip section configured in accordance with various embodiments of the present technology.

FIG. 8B is a partially schematic rear view of the tip section of FIG. 8A.

FIG. 9 is a partially schematic, transparent side view of another tip section configured in accordance with various embodiments of the present technology.

FIG. 10 is a partially schematic, transparent side view of another tip section configured in accordance with various embodiments of the present technology.

FIG. 11 is a flow diagram illustrating a method in accordance with various embodiments of the present technology.

FIGS. 12A and 12B are partially schematic, partially transparent side views of another tip section configured in accordance with various embodiments of the present technology.

FIGS. 13A and 13B are partially schematic views of an ablation electrode of the tip section of FIGS. 12A and 12B and configured in accordance with various embodiments of the present technology.

### DETAILED DESCRIPTION

The present disclosure is generally directed to ablation catheters with segmented energy delivery elements (e.g., ablation electrodes) and/or energy delivery elements having adjustable apertures. In some embodiments disclosed herein, an ablation catheter includes (i) a shaft having a proximal end and a distal end opposite the proximal end, and (ii) an ablation electrode at the distal end of the shaft. The ablation electrode can include a plurality of (e.g., ring-shaped) conductive segments centered about a common axis. Each segment can be independently connected to and/or independently energized by one or more energy sources. In these embodiments, any combination of the segments can be energized by either one energy source or multiple energy sources.

The segments of the ablation electrode can be spaced and insulated from each other, and can be held together by an insulating envelope. In some embodiments, the segments can have different/varying radii. In embodiments incorporating planar or generally planar segments, the segments of the ablation electrode can be positioned such that their planes are arranged generally perpendicular to a common axis. The ablation electrode can be configured such that a most proximal segment is the smallest and a most distal segment is the largest. Continuing with this example, the plurality of segments can therefore enclose (or generally form) a concave dome with an oculus at its apex. When one or more of the segments is/are energized, the oculus can have a size corresponding to a most proximal energized segment, and an ablating current can be delivered to target tissue from an inner surface of the dome to ablate the tissue. Segments of the dome that are not energized can be left electrically floating and therefore electrically absent from the ablation electrode.

In some embodiments, a subset of the segments can be energized. As discussed above, a most proximal segment in the energized subset can define the effective oculus of the dome. Thus, by selecting which of the segments to include in the energized subset, an oculus size for the dome can be selected (e.g., controlled, adjusted, tailored), meaning that an electric field distribution in target tissue can be selected (e.g., controlled, adjusted, tailored). As such, the present technology facilitates controlling (e.g., selecting, adjusting, tailoring) various characteristics (e.g., size, depth) of ablation lesions formed in target tissue.

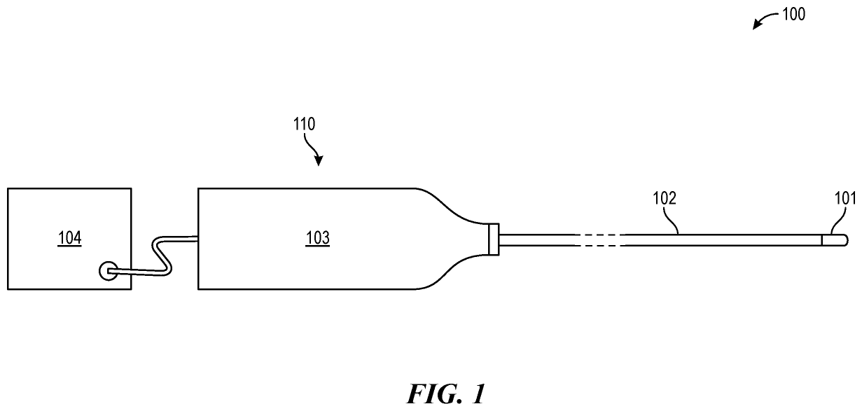

Specific details of several embodiments of the present technology are described herein with reference to FIGS. 1-13B. Although many of the embodiments are described with respect to devices, systems, and methods of applying ablative energy to tissue in a heart of a patient (e.g., for treating an arrhythmia), other applications and other embodiments in addition to those described herein are within the scope of the present technology. For example, unless otherwise specified or made clear from context, the devices, systems, and methods of the present technology can be used for any of various medical procedures, such as procedures performed on a hollow anatomical structure of a patient or on other anatomical structures (e.g., tumors).

It should be noted that other embodiments in addition to those disclosed herein are within the scope of the present technology. Further, embodiments of the present technology can have different configurations, components, and/or procedures than those shown or described herein. Moreover, a person of ordinary skill in the art will understand that embodiments of the present technology can have configurations, components, and/or procedures in addition to those shown or described herein and that these and other embodiments can be without several of the configurations, components, and/or procedures shown or described herein without deviating from the present technology.

As used herein, the term “physician” shall be understood to include any type of medical personnel who may be performing or assisting a medical procedure and, thus, is inclusive of a doctor, a nurse, a clinician, a medical technician, other similar personnel, and any combination thereof. As used herein, the term “patient” should be considered to include human and/or non-human (e.g., animal) subjects upon which a medical procedure is being performed.

### A. Overview

Focused Electric Field (FEF) ablation is an ablation technique that can be employed to ablate tissues much deeper than with conventional ablation catheters. FEF ablation is based on the principle that the pattern (or distribution) of a static (or quasi static) electric field depends on the geometrical distribution of electric charges that cause the field. For example, in a capacitor made of two large, parallel metal plates, the field between the plates) will be uniform as a result of uniform distribution of charges on the conductive surfaces. Charging a conductive non-planar surface, however, will provide a non-uniform electric field. More specifically, electric field lines in the proximity of a convex surface will diverge, and electric field lines in the proximity of a concave surface will converge.

Unlike in traditional ablation techniques, FEF ablation uses an ablation electrode having a concave structure or feature (e.g., a partial dome shape) and, optionally, dielectric insulation along the sides of the ablation electrode and on its rim. The concave surface can be continuous, or it can be formed of/defined by multiple segments that are insulated from one another. The entire surface (the entire continuous surface or the entire surface formed of/defined by the multiple segments) can be uniformly charged, or at least one of the segments can have a different amount of charge than another of the segments. Energy can be delivered to tissue from within the concave conductive surface of the ablation electrode. Such a design can suppress undesired, strong fields associated with shapes (e.g., edges, rims) that have small radii of curvature. Such a design is also expected to lead to a markedly different electric field distribution than conventional techniques, with a much slower fall-off of the electric field with distance from the ablation electrode. This more uniform electric field is expected to allow for deeper ablation while reducing the risk of excessive tissue heating and steam pops during ablation. FEF ablation electrodes can be used to deliver energy either as continuous, radio frequency (RF) power or as a series of short, high-power pulses. The series of pulses can be used for pulsed-field ablation (PFA). Other uses or modalities consistent with the disclosure are also possible. Whether FEF technology is used in conjunction with RF energy or with emerging technologies such as PFA or electroporation, the ability to deliver electrical energy deeper into the tissue while avoiding superficial tissue heating and energy loss is essential to ablating arrhythmogenic foci at mid-myocardial or ventricular intramural sites. Additional details of FEF ablation catheters are disclosed in U.S. patent application Ser. No. 17/722,533, the disclosure of which is incorporated by reference herein in its entirety.

Several embodiments of the present technology disclosed in detail below are generally directed to FEF ablation catheters and/or other ablation catheters. For example, several embodiments of the present technology are directed to cardiac ablation catheters including one or more ablation electrodes that can be energized via, for example, energy supplied by an energy source. Energy supplied to the ablation electrodes can be applied to tissue at a delivery site on or within a patient to form lesions at the delivery site. More specifically, the ablation electrode can be energized by the energy source to generate an electric field that can be used to deliver energy to target tissue to ablate the tissue. The distribution of the electric field can influence shape, size, and/or other characteristics of lesions formed in the tissue. In other words, the shape, size, and/or other characteristics of lesions formed in tissue can be controlled by controlling the shape, size, and/or other characteristics of an ablating electric field generated by the ablation electrode.

The distribution of an electric field generated by the ablation electrode can depend on geometry, configuration, and/or arrangement of conducting parts of the ablation electrode. For example, the geometry, configuration, and/or arrangement of the conducting parts of the ablation electrode may influence the areas of ablated tissue and/or other characteristics (e.g., size, shape, depth) of lesions formed by applying energy to the tissue via the ablation electrode. Thus, modifying the geometry, configuration, and/or arrangement of conducting parts of the ablation electrode is expected to modify an electric field generated by those conducting parts and, as a result, lesions formed in tissue using the electric field.

As discussed in greater detail below, catheters configured in accordance with various embodiments of the present technology can employ ablation electrodes that each having a plurality of conductive segments (e.g., continuous, conductive regions or continuous, conductive components, and/or regions/components that, when energized, are brought to equal potentials at all points across conductive portions of those regions/components), all or a subset of which can be individually or collectively energizable to modify (e.g., select, adjust, tailor, alter, change) one or more characteristics of an electric field generated by that ablation electrode. For example, segmented ablation electrodes of the present technology can include a plurality of conductive cylinders, rings, or other shapes/volumes. As a specific example, segmented ablation electrodes of the present technology can include a stack of uniformly charged conductive (e.g., metallic) rings, such as one or more conductive rings that are concentric or share a central axis. In these embodiments, current delivered to target tissue from the ablation electrode may be delivered with symmetry respective to the rings arranged in space along a shared axis. Each of the rings can be (e.g., uniformly or unequally) spaced a specified distance from adjacent rings in the stack. In some embodiments, the rings can be identically or similarly sized (e.g., have a same diameter or similar diameters, such as a same average diameter or similar average diameters) and together form a cylindrically symmetric ablation electrode. In other embodiments the rings can be of varying sizes (e.g., have varying diameters or varying average diameters). For example, the rings can have increasing diameters or increasing average diameters moving from a proximal portion of the corresponding ablation electrode to a distal portion of the ablation electrode. As such, the rings of varying sizes can be stacked and together form certain shapes of ablation electrodes, such as domes, partial domes, or other concave-shaped ablating elements (e.g., having different electric field characteristics). Different combinations of the rings can be energized to produce different electric field profiles. In this manner, the present technology facilitates modifying (e.g., selecting, adjusting, tailoring, altering, changing) one or more characteristics of an electric field emitted by an ablation electrode and thereby modifying (e.g., selecting, adjusting, tailoring, altering, changing) the shape, size, depth, and/or other characteristics of lesions formed in target tissue via application of the electric field.

Additionally, or alternatively, catheters configured in accordance with various embodiments of the present technology can employ ablation electrodes (e.g., segmented ablation electrodes) having adjustable oculi (e.g., electrically adjustable oculi, mechanically adjustable oculi, magnetically adjustable oculi). In particular, the inventors of the present technology have determined that an oculus (sometimes also referred to herein as an “aperture”) at a top portion of an ablation electrode (e.g., a FEF ablation electrode) can affect a distribution of an electric field generated by the ablation electrode and applied to target tissue. For a dome-shaped, partially dome-shaped, or other concave-shaped ablation electrode, the terms “oculus” and “aperture” can refer to an opening or a hole in the ablation electrode. For example, an oculus may be an opening in the center or top of a dome-shaped ablation electrode. Additionally, or alternatively, depending on context, the terms “oculus” and “aperture” can refer to a smallest, two-dimensional region bounded by a most proximal edge of a most proximal energized segment of an ablation electrode. Continuing with this example, a size of such an oculus can be expressed as an area of the region or as an average radius of the region. An oculus/aperture may be of any shape, including ovular, elliptical, or circular shapes. As a non-limiting list, an oculus/aperture may have a circular, triangular, rectangular, hexagonal, or octagonal shape, or the like. As an example, an oculus/aperture may refer to a circular opening at an apex, or top, of a dome-shaped ablation electrode.

The oculus of an ablation electrode can be aligned with or offset from a central axis of the ablation electrode. For example, the oculus of a dome-shaped ablation electrode may be disposed off-center with respect to the dome shape, such as an oculus having a central axis which is at an angle to the central axis of the dome shape. In some embodiments, the oculus can have at least one predetermined dimension. For example, the predetermined dimension may refer to a (e.g., average) diameter, width, depth, or height of the opening, either measured absolutely or relative to the dome shape. In some embodiments, the size or effective size of the oculus is selectable. Selectable may refer to a capability of being chosen or changed. A selectable size may involve oculus sizes which may be predetermined or adjustable to a desired size. For example, the size of the oculus may be controlled and changed if desired, such as by adjusting the amount of opening via a mechanical iris or shutter and/or by energizing different combinations of conductive segments of a segmented ablation electrode. The size of an oculus may be selected before delivery to the target tissue or adjusted during delivery or during ablation by various sensors, processors, and/or controllers. In a specific example, the effective size of an oculus of a segmented ablation electrode can be selected by selecting which conductive segments of the segmented ablation electrode are connected to and/or supplied energy from an energy source. In this manner, the present technology facilitates changing one or more characteristics (e.g., size or diameter, such as average size or average diameter) of oculi/apertures in ablation electrodes to modify electric fields generated by the ablation electrodes. Because size, shape, depth and/or other characteristics of lesions formed in target tissue via application of electric fields depends at least in part on various characteristics of the electric fields, such control over the various characteristics of the electric fields generated by the ablation electrodes is expected to enhance the ablating function of catheters configured in accordance with the present technology.

According to some additional aspects of the present disclosure, FEF ablation can be performed in a perpendicular direction with respect to a major (longitudinal) axis of a catheter shaft, in a parallel direction with respect to the major axis, and/or at a variety of other angles with respect to the major axis. For example, when a FEF ablation electrode configured in accordance with the present technology is employed to ablate tissue at various angles, the FEF effect is substantially preserved up to an angle of 45 degrees from the longitudinal axis of the catheter shaft with little to no loss of depth in observed ablation lesions. More specifically, computational analysis of an electric field generated by a FEF ablation electrode confirms that the electric field does not decay as quickly with distance as initially expected, thereby allowing for deeper tissue ablation. Additional experimental work demonstrates that there is a relative angle-independence of the FEF effect at off-axis catheter orientations. For example, Table 1 below summarizes the results obtained from two in vivo ablations, demonstrating no loss of lesion depth up to 45 degrees.

TABLE 1

| | Depth (mm) | Width (mm) |
|---|---|---|
| All lesions | 18 ± 4.3 | 23 ± 6.1 |
| 45 degrees | 18.5 ± 4.4 | 22.75 ± 8.5 |
| 60-90 degrees | 18 ± 5.3 | 21.5 ± 7.0 |

Thus, the inventors of the present technology have determined that an electric field generated by a FEF ablation electrode (e.g., a dome-shaped, partially dome-shaped, and/or concave-shaped ablation electrode) remains confined to a narrow tissue region, thereby avoiding rapid fall-off in energy delivery from the tissue surface that is inherent to conventional ablation systems. As such, FEF ablation according to the current disclosure is expected to advantageously allow significantly deeper ablation than current ablation technologies, with an improved safety margin.

### B. Selected Embodiments of Cardiac Ablation Catheters with Segmented Energy Delivery Elements Having Adjustable Apertures, and Associated Systems, Devices, and Methods

FIG. 1 is a partially schematic diagram of a catheter ablation system 100 (“the system 100”) configured in accordance with various embodiments of the present technology. As shown, the system 100 includes a catheter 110 having a shaft 102, a handle 103 at a proximal end portion of the shaft 102, and a tip section 101 at a distal end portion of the shaft 102. The handle 103 can be used to manipulate the shaft 102 and/or the tip section 101, such as to deliver the tip section 101 to a desired delivery site (e.g., within a heart of a patient). In other embodiments, the handle 103 can be omitted, such as in embodiments adapted for robotically assisted procedures. Additionally, or alternatively, although shown at a distal end portion of the shaft 102, the tip section 101 (or one or more components thereof) can be located at other locations along the shaft 102 or on other structures known in the art for accessing or treating tissue sites within or on a body of a patient.

In some embodiments, the shaft 102 and/or the tip section 101 can include a chamfer or a sloping surface, such as a sloping edge at a distal tip portion. The chamfer can include angled edges that may (i) provide easier navigation through blood vessels and/or (ii) prevent kinking or twisting of the shaft 102. In some embodiments, the chamfered edge can influence (a) a shape of an electric field generated by an ablation electrode (not shown) of the catheter 110 and/or (b) performance of the catheter 110 during an ablation procedure.

In the illustrated embodiment, the system 100 further includes an energy source 104 electrically connectable to the catheter 110. Electrically connectable may refer to being electrically coupled, such as to an electrical connection that permits the flow of current. Electrically connecting components may include employing conductors (e.g., wires, cables, soldering, integrated circuits) that permit current flow between the components. The energy source 104 can include sources of energy (e.g., RF energy, PF energy, or other electrical energy), such as generators, cells, batteries, capacitors, transformers, and/or voltage sources. Thus, at least when the energy source 104 is electrically connected to the catheter 110, the energy source 104 can be configured to supply energy (e.g., electrical energy, radiofrequency (RF) energy, pulsed-field (PF) energy such as for PFA or other ablation therapies) to various sensors, electrodes, microprocessors, and/or other electrical devices disposed on or in the catheter 110 (e.g., at the tip section 101 or elsewhere along the shaft 102). The energy source 104 can be configurable to deliver power, voltage, and/or current to the catheter 110 with time-dependent amplitude.

Energy supplied to the catheter 110 by the energy source 104 can be provided to an ablation electrode (e.g., at the tip section 101) of the catheter 110 and thereby applied to target tissue at a delivery site on or within a patient. More specifically, the ablation electrode can be energized by the energy source 104 to generate an electric field that is useable to deliver energy to target tissue to ablate such tissue. Data corresponding to energy delivered to the ablation electrode and/or to tissue may be collected from the catheter 110 or the ablation electrode. For example, data corresponding to energy supplied to the ablation electrode from the energy source 104 may be collected via sensors, circuit implementations (e.g., voltage detection circuits), or microcontrollers. As another example, data corresponding to an energy profile (e.g., a distribution of electrical energy in tissue, which, in some cases, can be coincident with a profile of a lesion formed in tissue via application of the electrical energy to the tissue) or electric field applied or delivered to tissue via the ablation electrode may be collected. In some embodiments, data corresponding to (i) energy supplied to the ablation electrode from the energy source 104 and/or (ii) an energy profile or electric field applied or delivered to tissue via the ablation electrode, may be used to provide a feedback loop (e.g., to inform and/or control future energy delivery to the ablation electrode and/or to tissue) and/or to generate analytics.

As discussed above, the distribution of an electric field generated by an ablation electrode can influence shape, size, and/or other characteristics of lesions formed in tissue. In other words, the shape, size, and/or other characteristics of lesions formed in tissue can be controlled by controlling the shape, size, and/or other characteristics of an ablating electric field generated by the ablation electrode. The distribution of an electric field generated by the ablation electrode can depend on geometry, configuration, and/or arrangement of conducting parts of the ablation electrode. For example, the geometry, configuration, and/or arrangement of the conducting parts of the ablation electrode may influence the areas of ablated tissue and/or other characteristics (e.g., size, shape, depth) of lesions formed in tissue by applying energy to the tissue via the ablation electrode. Thus, modifying the geometry, configuration, and/or arrangement of conducting parts of the ablation electrode is expected to modify an electric field generated by those conducting parts and, as a result, lesions formed in tissue using the electric field.

Therefore, as discussed in greater detail below, the catheter 110 and/or other catheters configured in accordance with the present technology can employ segmented ablation electrodes having a plurality of conductive segments (e.g., conductive regions, conductive components, conductive parts, conductive portions, conductive structures) that can be energized individually, collectively, and/or in subgroups to alter (e.g., adjust, tailor, change, modify) one or more characteristics of an electric field generated by the segmented ablation electrodes and thereby alter (e.g., select, adjust, tailor, change, modify) one or more characteristics of lesions formed in tissue using the electric field. For example, segmented ablation electrodes of the present technology can include a plurality of conductive cylinders, rings, or other shapes/volumes. As a specific example, segmented ablation electrodes of the present technology can include a stack of uniformly charged conductive (e.g., metallic) rings, such as one or more conductive rings that are concentric or share a central axis. Each of the rings can be (e.g., uniformly or unequally) spaced a specified distance from adjacent rings in the stack. In some embodiments, the rings can be identically or similarly sized (e.g., have a same diameter/average diameter or similar diameters/average diameters) and together form a cylindrically symmetric ablation electrode. In other embodiments the rings can be of varying sizes (e.g., have varying diameters/average diameters). For example, the rings can have increasing diameters/average diameters moving along an ablation electrode from a proximal portion of the ablation electrode to a distal portion of the ablation electrode. As such, the rings of varying sizes can be stacked and together form certain shapes of ablation electrodes, such as domes, partial domes, or other concave-shaped ablating elements (e.g., having different electric field characteristics).

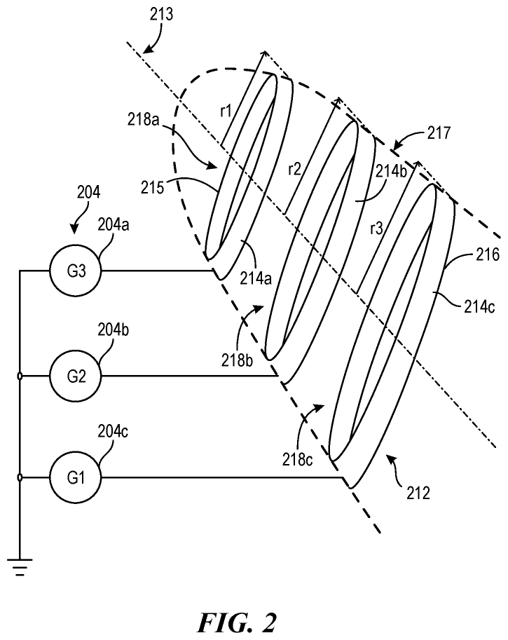

FIG. 2, for example, is a partially schematic, side perspective view of a segmented ablation electrode 212 configured in accordance with various embodiments of the present technology. As shown, the segmented ablation electrode 212 includes conductive segments 214 that are identified individually in FIG. 2 as first conductive segment 214a, second conductive segment 214b, and third conductive segment 214c. In the illustrated embodiment, the conductive segments 214a-214c are conductive rings of varying sizes (e.g., varying radii or diameters, such as varying average radii or varying average diameters). For example, the conductive segments 214a-214c can generally be (or be shaped as) conic sections having a first (e.g., distal) radius that is larger than a second (e.g., proximal) radius. The conductive segments 214a-214c are generally stacked or arranged about a common axis such that a general shape of the segmented ablation electrode 212 is a partial dome 217 that is at least partially electrically conductive and that has a central axis 213. The term ‘partial dome’ as used herein refers to a curved formation or structure, such as a half sphere or a semi-sphere. For example, a partial dome may include an ovular or circular circumference and/or may include a circumference that increases from one end of the partial dome shape (e.g., an apex) to the other end (e.g., an opening end). For example, in the illustrated embodiment, the conductive segments 214a-214c are concentric and/or arranged such that the radii (e.g., the proximal radii) of the conductive segments 214a-214c increase from a proximal end 215 of the segmented ablation electrode 212 to a distal end 216 of the segmented ablation electrode 212. More specifically, a radius r1 of the first conductive segment 214a can be smaller than a radius r2 of the second conductive segment 214b and a radius r3 of the third conductive segment 214c, and the radius r2 of the second conductive segment 214b can be smaller than the radius r3 of the third conductive segment 214c.

The partial dome 217 formed by the conductive segments 214a-214c includes an apex (or top side) at the proximal end 215 of the segmented ablation electrode 212, and an opening side at the distal end 216 of the segmented ablation electrode 212. The apex and/or the opening side may be circular, ovular, elliptical, or have a shape of a ring or rim. As discussed in greater detail below, when the segmented ablation electrode 212 is positioned within a catheter shaft, such as within the tip section 101 (FIG. 1) of the catheter 110 (FIG. 1), the rim at the opening side of the partial dome 217 (e.g., at the distal end 216 of the segmented ablation electrode 212) can be covered by an insulating material (e.g., rubber, plastic). Additionally, or alternatively, all or a subset of the partial dome 217 may be surrounded on its outer side (e.g., on its non-ablating side) by an insulating material (not shown). In other embodiments, the apex and/or the opening can have a different shape than shown, such as a non-circular or non-ovular shape.

All or a subset of one or more of the conductive segments 214a-214c and/or all or a subset of the ablation electrode 212 can be formed of conductive material. For example, all of the first conductive segment 214a, the second conductive segment 214b, and/or the third conductive segment 214c can be formed of a conductive material such that the first conductive segment 214a, the second conductive segment 214b, and/or the third conductive segment 214c is/are uniformly conducting or is/are uniformly chargeable. As another example, only a portion of the first conductive segment 214a, the second conductive segment 214b, and/or the third conductive segment 214c can be formed of a conductive material such that only the portion of the first conductive segment 214a, the portion of the second conductive segment 214b, and/or the portion of the third conductive segment 214c is/are conducting or chargeable.

In some embodiments, all or a subset of one or more of the conductive segments 214a-214c and/or all or a subset of the ablation electrode 212 can be formed of a mesh material. For example, all or a subset of the conductive segments 214a-214c, a cover (not shown) about the ablation electrode 212, and/or a structure (not shown) connecting the conductive segments 214a-214c to a catheter shaft may formed of a mesh material (e.g., a conductive mesh material). As used herein, the term “mesh” can refer to a woven material, such as materials produced by twisting, interlocking, or knitting. Further, the term mesh may include materials such as fibers, metals (e.g., stainless steel, copper, bronze, etc.), and/or plastics (e.g., polyester, nylon, polypropylene, etc.). A mesh material may be conductive by including metals or other materials capable of conducting electricity. In some embodiments, the term mesh can include foldable mesh materials (e.g., conductive foldable mesh materials). Foldable mesh materials may refer to flexible mesh materials that are capable of changing shape or configuration. For example, all or a subset of one or more the conductive segments 214a-214c and/or all or a subset of the segmented ablation electrode 212 can be formed of a foldable mesh material that (i) enables retracting the ablation electrode 212 into a catheter shaft (not shown), such as during delivery or navigation to target tissue, and/or (ii) enables expanding the ablation electrode 212 (e.g., from within a catheter shaft), such as upon arrival at target tissue for ablation. In some embodiments, mesh materials used to form the conductive segments 214a-214c and/or other portions of the ablation electrode 212 can be lightweight and flexible.

As shown in FIG. 2, the conductive segments 214a-214c are separated from each other by a distance. For example, the conductive segments 214a-214c can be uniformly or non-uniformly spaced apart from one another. As a specific example, the second conductive segment 214b can be spaced apart from the first conductive segment 214a by a first distance (e.g., approximately 1 mm), the third conductive segment 214c can be spaced apart from the second conductive segment 214b by a second distance (e.g., approximately 1 mm), and/or the third conductive segment 214c can be spaced apart from the first conductive segment 214a by a third distance (e.g., approximately 2 mm).

Distances between the conductive segments 214a-214c can be fixed or variable. For example, as discussed in greater detail below, the distance between the first conductive segment 214a and the second conductive segment 214b and/or the distance between the conductive segment 214 and the third conductive segment 214c can be changed (e.g., modified, adjusted, tailored, selected, altered) in some embodiments. As a specific example, insulated regions positioned between immediately adjacent ones of the conductive segments 214a-214c can be compressed or expanded. As another example, an amount of the insulated regions positioned between immediately adjacent ones of the conductive segments 214a-214c can be modified. In turn, the distances between the immediately adjacent ones of the conductive segments 214a-214c can be changed.

As discussed in greater detail below, the segmented ablation electrode 212 can include insulated regions (not shown). The insulated regions can be positioned between immediately adjacent ones of the conductive segments 214a-214c, within an interior of or a volume surrounded by the partial dome 217, and/or about a perimeter of the partial dome 217. In these and other embodiments, at least some of the insulated regions can hold the conductive segments 214a-214c within a catheter shaft and/or in the arrangement shown in FIG. 2. The insulated regions may include regions of low or no electrical conductivity. For example, the insulated regions may include insulators, such as rubber or plastic. Thus, the insulated regions may assist in electrically insulating individual ones of the conductive segments 214a-214c from one another, and/or in generating or realizing independent or distinct electrical distributions from each of the conductive segments 214a-214c, as discussed in greater detail below.

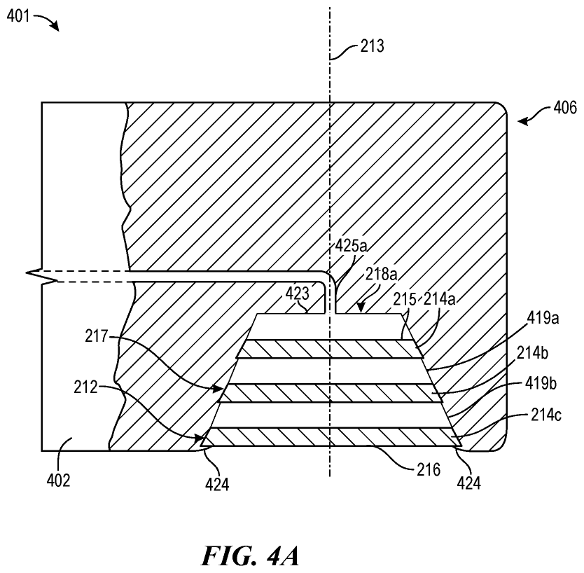

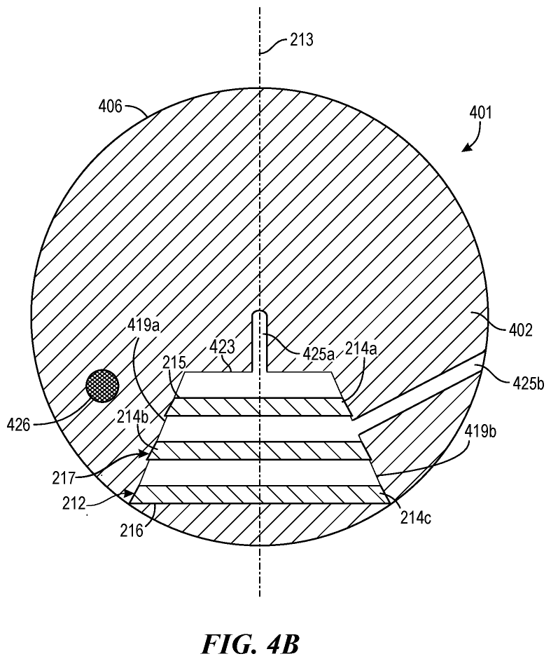

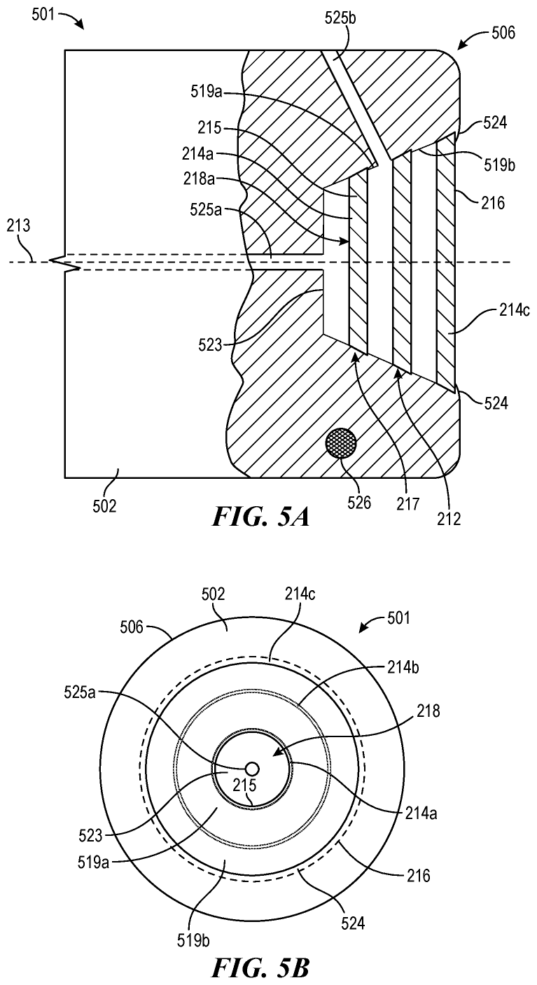

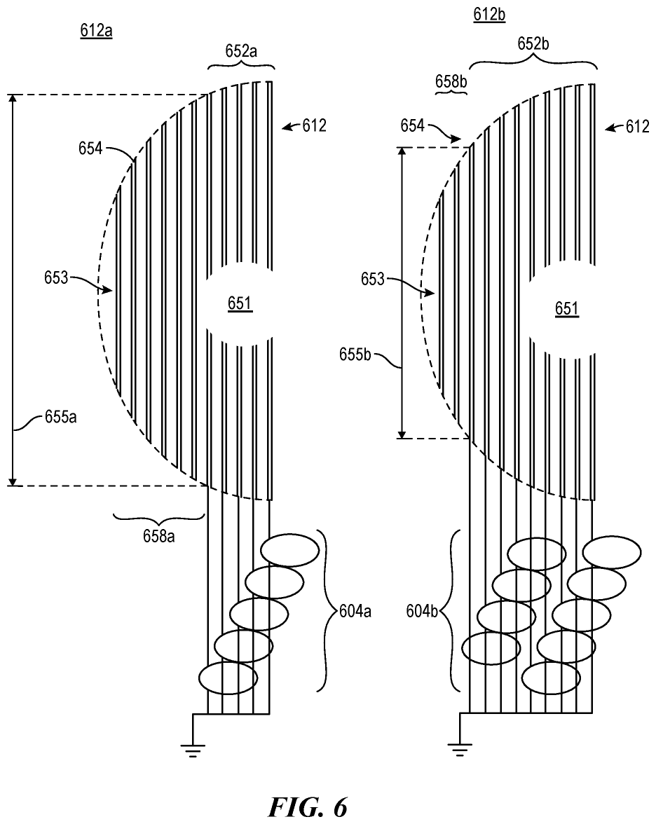

As discussed in greater detail below with reference to FIGS. 4A and 4B, the segmented ablation electrode 212 can be arranged (e.g., positioned on or in a catheter, such as on or in the tip section 101 of the catheter 110 of FIG. 1) such that the central axis 213 of the partial dome 217 is generally perpendicular to a major (or longitudinal) axis of a catheter shaft (e.g., the shaft 102 of FIG. 1). For example, the orientation of the partial dome 217 formed by the segmented ablation electrode 212 may be at an angle of 90 degrees relative to the longitudinal axis of a catheter shaft. As discussed in greater detail below with reference to FIGS. 5A and 5B, the segmented ablation electrode 212 can alternatively be arranged such that the central axis 213 of the partial dome 217 is generally parallel to the longitudinal axis of a catheter shaft. In still other embodiments, the segmented ablation electrode 212 can be arranged such that the central axis 213 of the partial dome 217 is at a non-parallel and/or non-perpendicular angle relative to the longitudinal axis of the catheter shaft, or may be adjusted by a shift relative to the longitudinal axis and remain parallel to the longitudinal axis. In these and still other embodiments, an angle of the central axis 213 of the partial dome 217 relative to the longitudinal axis of the catheter shaft can be adjusted (e.g., in vivo or before insertion of the catheter into a patient). It is appreciated that different orientations of the partial dome 217 of the segmented ablation electrode 212 (e.g., perpendicular, parallel, or angled relative to the longitudinal axis of the catheter shaft) may assist in providing electric fields which ablate deeper tissues or tissues in difficult positions.

Referring again to the embodiment illustrated in FIG. 2, the conductive segments 214a-214c can be electrically connected to one or more energy sources 204. The energy source(s) 204 can be example(s) of the energy source 104 of FIG. 1 and/or other energy sources of the present technology. The energy source(s) 204 can be sources of electrical energy (e.g., RF or PF energy) and/or can be used to modulate power, voltage, and/or current supplied to corresponding conductive segments 214a-214c of the segmented ablation electrode 212.

In some embodiments, at least one of the conductive segments 214a-214c is independently connectable to the energy source(s) 204. Independently connectable may refer to electrical connections which may not necessarily affect or depend upon other electrical connections. For example, the first conductive segment 214a can be independently connectable to a first energy source 204a such that the first conductive segment 214a can be individually energized via the first energy source 204a. Additionally, or alternatively, the first conductive segment 214a can be independently connectable to the first energy source 204a such that the first energy source 204 can modulate power, voltage, or current supplied to the first conductive segment 214a without affecting (i) the electrical energy output by a second energy source 204b and/or a third energy source 204c and/or (ii) characteristics of electrical energy supplied to the second conductive segment 214b and/or to the third conductive segment 214c. As a specific example, the first energy source 204a can modulate power, voltage, or current supplied to the first conductive segment 214a while power, voltage, or current supplied to the second conductive segment 214b and/or the third conductive segment 214c remain constant. In these and other embodiments, the second conductive segment 214b can be independently connectable to the second energy source 204b, and/or the third conductive segment 214c can be independently connectable to the third energy source 204c. For example, while the first conductive segment 214a and the second conductive segment 214b are not energized and/or are not used to deliver energy to tissue, the third conductive segment 214c (representing a distalmost conductive segment of the segmented ablation electrode 212 in the illustrated embodiment) can be energized and/or used to deliver energy to tissue. In these and still other embodiments, two or more of the conductive segments 214a-214c can be connectable to a common energy source, and/or can be energized together (e.g., at a same time and/or via common electrical connections) via a same energy source 204 or different energy sources 204. In this manner, all or any subset (e.g., every one of or any fraction of) the conductive segments 214a-214c of the segmented ablation electrode 212 can be simultaneously or sequentially energized (e.g., to deliver energy to tissue).

As shown in FIG. 2, the partial dome 217 formed by the segmented ablation electrode 212 incudes various apertures or oculi. For example, the partial dome 217 includes a first aperture 218a (e.g., a first oculus) at the apex of the partial dome 217 (e.g., at the proximal end 215 of the segmented ablation electrode 212 corresponding to a proximalmost portion of the first conductive segment 214a in the illustrated embodiment). Continuing with this example, the partial dome 217 includes (i) a second aperture 218b (e.g., a second oculus) at a proximalmost portion of the second conductive segment 214b and (ii) a third aperture 218c (e.g., a third oculus) at a proximalmost portion of the third conductive segment 214c. Given that the conductive segments 214a-214c can be individually or collectively energizable in any subset of the conductive segments 214a-214c, the effective size of the aperture of the segmented ablation electrode 212 can be adjusted by varying which of the conductive segments 214a-214c are energized at a given time. For example, by energizing (i) the first conductive segment 214a and/or (ii) the first conductive segment 214a in combination with the second conductive segment 214b and/or the third conductive segment 214c, the segmented ablation electrode 212 can have an effective aperture (e.g., an effective oculus) corresponding to the first aperture 218a. In contrast, by not energizing the first conductive segment 214a while energizing (a) the second conductive segment 214b and/or (b) the second conductive segment 214b with the third conductive segment 214c, the segmented ablation electrode 212 can have a larger effective aperture corresponding to the second aperture 218b. In other words, the effective aperture and/or the effective size the aperture of the partial dome 217 of the segmented ablation electrode 212 can be altered (e.g., adjusted, tailored, changed, modified, selected) based on which of the conductive segments 214a-214c are energized at a given time. As discussed in greater detail below, it is expected that altering the effective aperture and/or the effective size of the aperture of the segmented ablation electrode 212 alters one or more characteristics (e.g., size, shape, focal depth, etc.) of an electric field generated by the segmented ablation electrode 212. Thus, it is expected that altering the effective aperture and/or the effective size of the aperture of the segmented ablation electrode 212 can alter one or more characteristics (e.g., size, shape, depth, etc.) of lesions formed in tissue using the electric field generated by the segmented ablation electrode 212.

In some embodiments, at least one of the conductive segments 214a-214c is configured to produce or emit a different energy profile. An energy profile may refer to various electrical or physical characteristics corresponding to an ablation electrode 212 and/or to a conductive segment 214 of the ablation electrode 212. An energy profile may include representations of an electrical field, such as an electric field produced or generated by a conductive segment 214 and/or by an ablation electrode 212. Conductive segments 214 may produce or emit different energy profiles when electrically connected to different energy sources or by varying onboard electronics (e.g., resistors, capacitors, diodes, inductors, transistors). Energy profiles produced or generated by conductive segments 214 may depend on circuit configurations connecting the conductive segments 214 to corresponding energy sources 204. For example, a conductive segment 214 may produce different energy profiles when connected to an energy source 204 via parallel or series configurations and/or when employing differing passive or active circuit components. Thus, it is expected that an energy profile of a conductive segment 214 and/or of an ablation electrode 212 can be modified by modifying the corresponding circuit connections and/or circuit components used to connect that conductive segment 214 to a corresponding energy source 204. As another example, the energy profile produced or generated by an ablation electrode 212 may also be varied by adjusting different conductive segments 214 of the ablation electrode 212, such as switching power to certain conducive segments 214 from on to off. In some embodiments, the different energy profile of a conductive segment 214 and/or of an ablation electrode 212 can be manipulated to adjust an energy pattern delivered by the ablation electrode 212. Energy patterns may include electric fields, distributions of current, amplitudes of power, and shapes or directions of delivery. By manipulating an energy profile of a conductive segment 214 and/or of an ablation electrode 212, an energy pattern delivered to tissue via the ablation electrode 212 may vary (e.g., a distribution of an electric field delivered to tissue may change). For example, by changing energy supplied to different conductive segments 214 of an ablation electrode 212, an energy profile of the ablation electrode 212 may change, which may result in a corresponding change in an electric field generated by the ablation electrode 212 and/or delivered to tissue. It will be appreciated that using different energy profiles, such as different electric fields delivered from the ablation electrode 212, may provide the benefit of controlling the shape of the ablating field and thereby control the shape, size, or depth of lesions formed in ablated tissue.

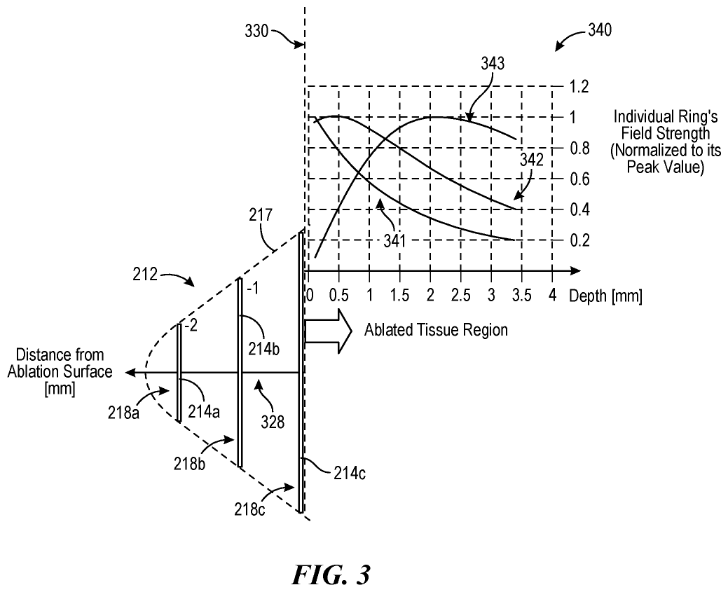

FIG. 3 is a partially schematic side view of the ablation electrode 212 of FIG. 2 and of a line plot 340 in accordance with various embodiments of the present technology. A blood-tissue boundary 330, such as a wall of a heart of a patient, is also shown in FIG. 3. In the illustrated embodiment, the ablation electrode 212 is oriented perpendicular to and contacting the boundary 330. A distance axis 328 is provided for the sake of clarity and example. In particular, the conductive segments 214a-214c are spaced 1 mm apart from one another in the illustrated embodiment such that the third conductive segment 214c is positioned 0 mm away from the boundary 330, the second conductive segment 214b is positioned 1 mm away from the boundary 330, and the first conductive segment 214a is positioned 2 mm away from the boundary 330. As discussed above, the distances between the conductive segments 214a-214c can be fixed or variable, can be different in other embodiments of the present technology from those distances shown in FIG. 3, and/or can be non-uniform (e.g., such that the distances between the boundary 330 and one or more of the conductive segments 214a-214c can differ from those shown in FIG. 3).

As discussed above, each of the conductive segments 214a-214c may be connected to an energy source (not shown in FIG. 3). For example, the first conductive segment 214a, the second conductive segment 214b, and the third conductive segment 214c may each be electrically connected to different energy sources whose output levels may be individually adjusted. In some embodiments, a single energy source may have multiple output channels which each may be electrically connected to a corresponding one of the conductive segments 214a-214c. In an additional example, output channels from a single energy source may be dependently connected to one or more of the conductive segments 214a-214c.

Referring to the line plot 340, electric field strength (normalized to peak value) from each of the conductive segments 214a-214c when energized individually is shown as a function of ablation depth (in mm) into tissue. For example, the line plot 340 includes a first curve 341 corresponding to the first conductive segment 214a, a second curve 342 corresponding to the second conductive segment 214b, and a third curve 343 corresponding to the third conductive segment 214c. As shown, the conductive segments 214a-214c each have different energy profiles, as seen in how the electric field strengths change as a function of depth into the tissue.

As discussed above, the ablation electrode 212 may be operated such that the first conductive segment 214a, the second conductive segment 214b, and the third conductive segment 214c may each be energized alone or in any combination, in order to achieve a desired field strength distribution in tissue. More specifically, each of the conductive segments 214a-214c may, when energized individually, provide a separate and distinct electric field. Additionally, or alternatively, each of the conductive segments 214a-214c may, when energized in a group including at least one of the other conductive segments 214a-214c, contribute to a combined or total electric field representing a vector sum of the individual electric field patterns generated by the individual conductive segments 214a-214c of the group. Thus, the conductive segments 214a-214c can be energized individually or in groups, thereby allowing control of the shape, size, depth, and/or other characteristics of an ablating field emitted by the segmented ablation electrode 212 and applied to target tissue, and consequently control of the shape, size, depth, and/or other characteristics of lesions formed in the target tissue. In other words, by modulating current or other electrical parameters supplied to the different conductive segments 214a-214c, an electric field pattern emitted by the segmented ablation electrode 212 can be controlled (e.g., selected, altered, changed, modified, tailored). For example, selection of which of the conductive segments 214a-214c to energize and/or selection of voltages or other energy parameters to supply to the selected conductive segments 214a-214c may allow control of the electric field distribution, and thereby control of lesion depth and width. It is therefore appreciated that using a larger number of conductive segments (e.g., more than one, more than three, etc.) may provide better control over the electric field at targeted depths in tissue. In some cases, it is expected that such control can provide the ability to fine-tune performance, such as by ablating at multiple depths, ablating at deeper depths, or providing a stronger electric field with certain groupings of the conductive segments 214a-214c.

In some embodiments, the electric field emitted by the segmented ablation electrode 212 may be modulated by varying the distances between the conductive segments 214a-214c. For example, changing the distance between the second conductive segment 214b and the third conductive segment 214c may result in different energy profiles corresponding to the second conductive segment 214b and/or the third conductive segment 214c than shown by the second curve 342 and/or the third curve 343, respectively, in the line plot 340. Thus, in some embodiments of the present technology, distances between immediately adjacent ones of the conductive segments 214a-214c can be modified to control or modify (e.g., alter, tailor, select) one or more characteristics of an electric field emitted by the segmented ablation electrode 212 and applied at one or more depths into target tissue.

FIG. 4A is a partially schematic, partially transparent side view of a catheter tip section 401 configured in accordance with various embodiments of the present technology, and FIG. 4B is a partially schematic, partially transparent end view of the catheter tip section 401 of FIG. 4A. The catheter tip section 401 can be the tip section 101 of FIG. 1, or another suitable tip section configured in accordance with various embodiments of the present technology. Referring to FIGS. 4A and 4B together, the tip section 401 employs the segmented ablation electrode 212 of FIGS. 2 and 3. More specifically, the segmented ablation electrode 212 is positioned or embedded within a cavity at a distal end portion 406 of a catheter shaft 402 such that the central axis 213 of the partial dome 217 formed by the conductive segments 214a-214c of the segmented ablation electrode 212 is oriented generally perpendicular to the major or longitudinal axis of the shaft 402. In the illustrated embodiment, the catheter tip section 401 includes a non-conducting surface 423 at or proximate the apex of the partial dome 217 (e.g., at or proximate the proximal end 215 of the segmented ablation electrode 212, such as at a location above or proximal to the proximal end 215 of the segmented ablation electrode 212 and/or the apex of the ablation electrode 212), such that a top central area, or ceiling, of the partial dome 217 (or of a hollow in the shaft 402 and in or around which the ablation electrode 212 is positioned) is non-conductive.

As shown, the conductive segments 214a-214c are arranged in a stack and spaced apart from one another. In particular, a first insulated region 419a separates the first conductive segment 214a from the second conductive segment 214b, and a second insulated region 419b separates the second conductive segment 214b from the third conductive segment 214c. In the illustrated embodiment, the insulated regions 419a and 419b can at least partially occupy a volume surrounded by the segmented ablation electrode 212. In other embodiments, the insulated regions 419a and/or 419b can be constrained to a perimeter of the segmented ablation electrode 212 (e.g., such that the insulated regions 419a and 419b separate the conductive segments 214a-214c but do not occupy the volume surrounded by the segmented ablation electrode 212). In at least some of these embodiments, the segmented ablation electrode 212 can define a hollow or empty cavity.

The tip section 401 includes a rim 424 at a distal end 216 of the segmented ablation electrode 212. In some embodiments, the rim 424 corresponds to (a) the most distal edge of the most distal conductive segment (e.g., the distalmost edge/portion of the conductive segment 214c) of the segmented ablation electrode 212, and/or (b) a portion of the ablation electrode 212 that is positioned closest to the tissue when ablating the tissue. The rim 424 can have a radius of curvature or chamfer, such as for example, a bead, a fillet, or a bevel. In some embodiments the rim 424 can be electrically insulated, thermally insulated, or both. As best shown in FIG. 4A, the tip section 401 can include a flow port 425a for introducing irrigation or coolant fluid into an interior of the segmented ablation electrode 212 (e.g., into the volume defined or surrounded by the segmented ablation electrode 212). As best shown in FIG. 4B, the tip section 401 can further include a flow port 425b for aspirating fluid from the interior of the segmented ablation electrode 212. The tip section 401 can additionally, or alternatively, include one or more temperature sensors 426 (FIG. 4B). A temperature sensor 426 can take the form of a thermistor, a thermocouple, or another type of sensor. In use, the segmented ablation electrode 212 can be heated by conduction from contact with heated tissue (e.g., rather than by being heated directly by RF energy delivered to the segmented ablation electrode 212). A temperature sensor 426 can be mounted in proximity to the segmented ablation electrode 212. Thus, the temperature sensor 426 can help to provide a proxy for the temperature of the tissue being ablated to help prevent overheating of the tissue. It is expected that overheating of the segmented ablation electrode 212 can lead to degraded performance and an increased risk of embolic stroke. A temperature measured below a certain threshold may also be an indication of poor or incomplete contact between the segmented ablation electrode 212 and the tissue, resulting in low heat conduction from the tissue to the tip section 401.

FIG. 5A is a partially schematic, partially transparent side view of another catheter tip section 501 configured in accordance with various embodiments of the present technology, and FIG. 5B is a partially schematic, partially transparent end view of the catheter tip section 501 of FIG. 5A. The catheter tip section 501 can be the tip section 101 of FIG. 1, or another tip section configured in accordance with various embodiments of the present technology. Referring to FIGS. 5A and 5B together, the tip section 501 employs the segmented ablation electrode 212 of FIGS. 2 and 3, and is generally similar to the tip section 401 of FIGS. 4A and 4B. Thus, similar reference numbers are used across FIGS. 4A-5B to denote generally similar components. In contrast with the tip section 401 of FIGS. 4A and 4B, however, the tip section 501 of FIGS. 5A and 5B employs the segmented ablation electrode 212 such that the segmented ablation electrode 212 is positioned or embedded within a cavity at a distal end portion 406 of a catheter shaft 402 with the central axis 213 of the partial dome 217 formed by the conductive segments 214a-214c of the segmented ablation electrode 212 oriented generally parallel with the major or longitudinal axis of the shaft 502.

Although shown as concentric conductive rings arranged in a stack with uniform spacing between the rings, the conductive segments 214a-214c of the segmented ablation electrode 212 of FIGS. 2-5B can have other shapes in other embodiments of the present technology, can have shapes that vary from one another, can be non-concentric (e.g., yet still arranged about a common axis), can be arranged side-by-side or in a different order than shown in FIGS. 2-5B, and/or can be spaced apart with a different (e.g., uniform or non-uniform) spacing in other embodiments of the present technology. For example, each of the conductive segments 214a-214c can have a same radius such that the ablation electrode 212 formed at least in part by the conductive segments 214a-214c is generally cylindrical. As another example, segmented ablation electrodes of other embodiments of the present technology can include any number of (e.g., less than three or more than three) conductive segments. As still another example, the conductive segments 214a-214c can be arranged in a different order than shown in FIG. 2 (e.g., such that the radii of the conductive segments 214a-214c do not increase from the proximal end 215 of the segmented ablation electrode 212 to the distal end of the segmented ablation electrode 212). In these and other embodiments, the segmented ablation electrode 212 can have a different shape other than the partial dome shape 217. In these and still other embodiments, at least one conductive segment can be differently shaped from another of the conductive segments of the segmented ablation electrode 212. For example, the third conductive segment 214c (representing a distalmost conductive segment in the embodiment illustrated in FIGS. 2-5B) can be generally cylindrical (e.g., as opposed to generally conical).

Additionally, or alternatively, ablation electrodes configured in accordance with other embodiments of the present technology can include non-circular apertures or oculi, and/or apertures/oculi that are disposed off-center with respect to the dome shape of the ablation electrode. For example, ablation electrodes configured in accordance with the present technology can include oculi having a central axis which is at an angle to the central axis of the dome shape. In some embodiments, an oculus of an ablation electrode can have at least one predetermined dimension. For example, the predetermined dimension may refer to a (e.g., average) diameter, width, depth, or height of the aperture, either measured absolutely or relative to the dome shape of the ablation electrode. As discussed above, the size of the oculus can be selectable or adjustable. Selectable may refer to a capability of being chosen or changed. A selectable size may involve oculus sizes which may be predetermined or adjustable to a desired size. For example, the size of the oculus may be controlled and changed if desired, such as by adjusting the amount of opening via a mechanical iris or shutter or by energizing select conductive segments of the ablation electrode. The size of the oculus may be selected before delivery to the target tissue or adjusted during delivery or during ablation by various sensors, processors, and/or controllers, or by selecting which conductive segments are connected to an energy source.

In these and other embodiments, an ablation catheter of the present technology can include one or more ablation electrodes (e.g., only one ablation electrode or a plurality of ablation electrodes) spaced along or about a catheter shaft. For example, ablation catheters of the present technology can include one or more segmented ablation electrodes and/or one or more ablation electrodes with adjustable apertures/oculi. Spaced along or about a catheter shaft may refer to ablation electrodes placed at different distances with respect to each other along the catheter shaft. Spaced along or about the catheter shaft may also refer to one or more ablation electrodes placed along or about the catheter shaft in different radial positions. In an example, the ablation electrodes may be placed at different distances to achieve a desired electric field distribution for ablation. Further, it will be recognized that a plurality of ablation electrodes may be positioned in various orientations with respect to a catheter shaft. For example, each of the plurality of ablation electrodes can have a central axis. The central axis of an ablation electrode may be an axis of symmetry for the ablation electrode. In an example, the central axis of one or more ablation electrodes may be parallel with respect to the longitudinal axis of the catheter shaft. In another example, the central axis of one or more ablation electrodes may be non-parallel (e.g., perpendicular or at another angle) with respect to the longitudinal axis of the catheter shaft. In some embodiments, one or more of the central axes of the plurality of ablation electrodes can be parallel to one another or at an angle to each other. As a specific example, an ablation catheter may have a plurality of at least partial dome shaped ablation electrodes spaced along the length of the catheter shaft, with the respective central axes of the partial domes (i) aligned parallel with one another, (ii) laying in parallel planes, and/or (iii) laying in planes that are non-parallel to one another. The ablation electrodes can reside in a single tip section of an ablation catheter, or in separate shafts.

As described herein, one or more ablation electrodes may include one or more conductive segments, such as one or more conductive rings connected to energy sources. The one or more conductive rings may be aligned with the central axis of the ablation electrode. In some embodiments, one or more of the central axes of the ablation electrodes can be non-parallel to one another. For example, the ablation electrodes may be oriented parallel or angled to one another. The orientation may be selected or adjusted based on a desired electric field distribution. As discussed herein, ablation electrodes may provide an electric field with varying strength at different tissue depths based on factors including energy source, configuration, energized conductive segments, distance between conductive segments, and aperture characteristics. Thus, disclosed embodiments may provide (i) ablation at various tissue depths and/or (ii) lesion shapes enabled by choosing various orientations for multiple ablation electrodes (and thereby choosing orientations of conductive segments within each electrode) to control electric fields. Additionally, or alternatively, as discussed above, ablation catheters of the present technology are usable to ablate tissue located lateral, perpendicular, or at another angle with reference to a major axis of the catheter shaft.

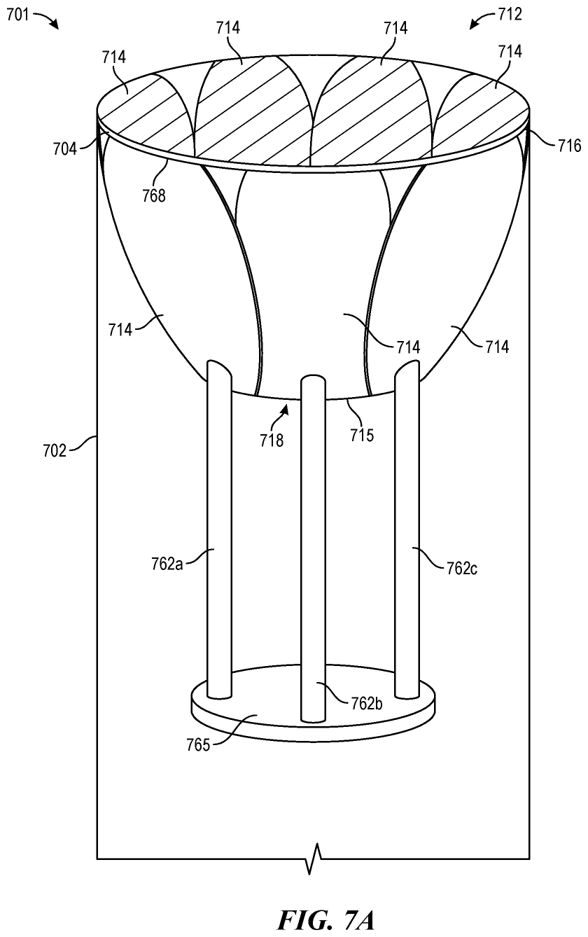

FIG. 6 is a partially schematic side view of another segmented ablation electrode 612 configured in accordance with various embodiments of the present disclosure. More specifically, FIG. 6 illustrates two scenarios 612a and 612b that correspond to two possible modes of energizing the segmented ablation electrode 612. As shown, the segmented ablation electrode 612 includes a plurality of conductive segments 651 (e.g., conductive rings) that are arranged to form a general dome shape 654. When all of the conductive segments 651 are energized, the segmented ablation electrode 612 can have an effective oculus 653 that corresponds to the smaller radius of the most proximal conductive segment 651 (e.g., to the most proximal or leftmost edge of the most proximal or leftmost conductive segment 651 in FIG. 6). Indeed, the segmented ablation electrode 612 can have the effective oculus 653 (i) in a scenario in which only the most proximal (or leftmost) conductive segment 651 of the ablation electrode 612 in FIG. 6 is energized, and/or (ii) in various scenarios in which the most proximal (or leftmost) conductive segment 651 of the ablation electrode 612 in FIG. 6 is energized in combination with all or a subset of the other conductive segments 651 of the ablation electrode.

Referring now to scenario 612a shown in FIG. 6, when conductive segments 651 of a first subset 652a are energized and all other conductive segments 651 of a second subset 658a are left electrically floating (or not energized) with no effect, the segmented ablation electrode 612 can have an effective oculus with a diameter 655a (corresponding to the smaller radius and/or the most proximal/leftmost edge of the most proximal/leftmost conductive segment 651 of the first subset 652a in FIG. 6). Energy sources 604a shown in FIG. 6 represent various energizing configurations for the conductive segments 651 of the first subset 652a, including a configuration in which all the conductive segments 651 of the first subset 652a are connected to one (e.g., a single, only one) source.

Referring now to scenario 612b shown in FIG. 6, when conductive segments 651 of a third subset 652b are energized and all other conductive segments of a fourth subset 658b are left electrically floating (or not energized) with no effect, the segmented ablation electrode 612 can have an effective oculus with a diameter 655b (corresponding to the smaller radius and/or the most proximal/leftmost edge of the most proximal/leftmost conductive segment 651 of the third subset 652b in FIG. 6). Energy sources 604b shown in FIG. 6 represent various energizing configurations for the conductive segments 651 of the third subset 652b, including a configuration in which all the conductive segments 651 of the third subset 652a are connected to one (e.g., a single, only one) source.

The scenarios described above with reference to FIG. 6 (including the scenarios 612a and 612b) therefore demonstrate that, by (i) selectively energizing a subset of the conductive segments 651 of the segmented ablation electrode 612 and (ii) setting operating parameters of the corresponding energy source(s) 604, an effective oculus of the segmented ablation electrode 612 can be controlled (e.g., selected, adjusted, tailored) that, in turn, can affect (e.g., control, select, adjust, tailor) characteristics of an energy profile generated by the segmented ablation electrode 612, for example, to control (e.g., select, adjust, tailor) characteristics (e.g., depth and/or size) of lesions formed in tissue via application of electrical energy from the segmented ablation electrode 612.

In some embodiments, the segmented ablation electrode 612 may be connected to a controller or microcontroller, as described herein. The controller may be configured to control the delivery of energy from the energy source(s) 604 to the conductive segments 651. For example, the controller may instruct the energy source(s) 604 to increase, decrease, turn on, turn off, or otherwise adjust energy delivered to the conductive segments 651. In some embodiments, the controller may follow a predetermined script or program to modulate the delivery of energy from the energy source(s) 604 to the conductive segments 651. As another example, the controller may automatically control the delivery of energy based on various sensors and feedback loops in the ablation catheter, as discussed herein. It will be appreciated that effective oculus (e.g., the diameter of the effective oculus) of the segmented ablation electrode 612 may thus be electronically controlled, thereby enabling disclosed embodiments to electronically adjust ablation depth in tissue during or in advance of an ablation procedure.

It will be recognized that an effective oculus of an ablation electrode may affect the distribution of an electric field in target tissue. In some embodiments, increasing the size of the effective oculus of an ablation electrode may produce deeper lesions in the ablated tissue. For example, with reference to FIG. 6, increasing the size of the effective oculus for the segmented ablation electrode 612 from the diameter 655b (scenario 612b) to the diameter 655a (scenario 612a) may result in ablating tissue at a deeper depth. In some embodiments, an ablation catheter may include various sensors to generate feedback for setting or adjusting (e.g., a size/diameter of) an effective oculus for a segmented ablation electrode, and/or for setting or adjusting power supplied to conductive segments of the ablation electrode. For example, sensors may be used to measure a size of an effective oculus of an ablation electrode and/or to generate a feedback loop to a corresponding controller or energy source. In turn, the controller/energy source can modulate voltage or another electrical parameter supplied to one or more conductive segments of the ablation electrode to, for example, control (e.g., set, adjust, tailor) depth of energy delivery into tissue and/or of lesions formed via application of electrical energy. In some embodiments, the feedback loop can include an operator of the ablation catheter, such as a physician. In some embodiments, the feedback loop may be automatic. For example, the ablation catheter may include sensors that can detect location and/or depth of target tissue and generate feedback to a corresponding controller or energy source to control (e.g., set, adjust, tailor) energy (e.g., power) supplied to one or more of the conductive segments of an ablation electrode, or to control (e.g., set, adjust, tailor) the size of an effective oculus of the ablation electrode, thereby adjusting depth, size, and/or other parameters of ablation, such as in accordance with a desired presurgical plan.

The inventors of the present technology analyzed an electric field for a segmented ablation electrode without an oculus versus an electric field of a segmented ablation electrode with an oculus. More specifically, the inventors plotted the distributions of the electric fields (in units of volts/centimeter) as a function of tissue depth (in millimeters). The plotted curve corresponding to the segmented ablation electrode with an oculus had a peak at a greater tissue depth than the plotted curve corresponding to the segmented ablation electrode without an oculus. As such, it is expected that segmented ablation electrodes with at least one oculus are capable of providing deeper ablation penetration than segmented ablation electrodes that lack an oculus.

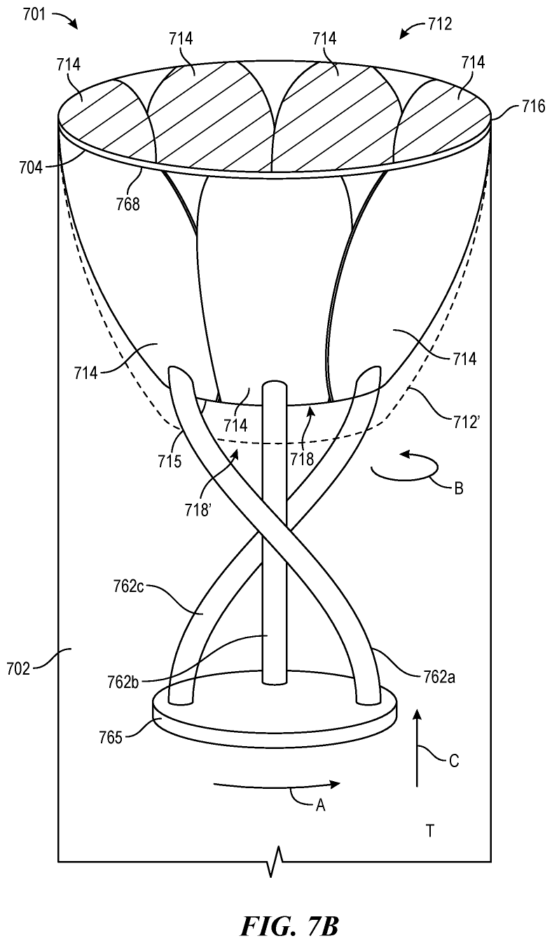

FIGS. 7A and 7B are partially schematic, transparent side views of another tip section 701 configured in accordance with various embodiments of the present technology. The tip section 701 can be an example of the tip section 101 of FIG. 1, or another tip section of the present technology. As shown, the tip section 701 includes a catheter shaft 702 and a segmented ablation electrode 712 positioned within the catheter shaft 702. The segmented ablation electrode 712 includes a plurality of conductive segments 714. In the illustrated embodiment, the conductive segments 714 are leaves (e.g., sections of one or more segments) that are arranged in a partially overlapping manner to give the segmented ablation electrode 712 a generally dome shape or a generally partial dome shape.

Each of the conductive segments 714 are captured (e.g., fixedly or slidably) at a distal end in a ring 768. For example, distal ends of the conductive segments 714 can be fixedly and/or slidably seated within the ring 768. In some embodiments, the ring 768 can be insulated and/or may include a slot or notch configured to receive the distal ends of the conductive segments 714. The ring 768 forms a rim 704 at a distal end 716 of the segmented ablation electrode 712 (and, in the illustrated embodiment, at a distal end of the catheter shaft 702). Each of the conductive segments 714 are further attached at a proximal end to a distal end of one of a plurality of rods 762, only three of which (identified individually in FIGS. 7A and 7B as first through third rods 762a-762c) are illustrated in FIGS. 7A and 7B for the sake of clarity. For example, each of the rods 762 can be pinned to a corresponding one of the conductive segments 714 such that they can rotate within a corresponding hole formed in the conductive segments 714. In turn, proximal ends of the rods 762 can be attached to a rotatable plate 765. The rods 762 can be generally flexible in some embodiments. Additionally, or alternatively, the rods can be connected directed or indirectly to a component (not shown) at a proximal end of the catheter shaft 702, such as to a handle (not shown), knob (not shown), or other actuation mechanism (not shown) that can be used to twist the rods 762 and/or the plate 765.

As shown in FIG. 7A, in a relaxed state, the segmented ablation electrode 712 includes an aperture or oculus 718 having a first size (e.g., a first diameter) at a proximal end 715 of the segmented ablation electrode 712. Referring now to FIG. 7B, the rotatable plate 765 can be twisted or turned generally along or parallel to the arrow A, which can twist the rods 762 generally along or parallel to the arrow B and apply a twisting force to shift or pivot the proximal ends of the conductive segments 714 in a same or similar direction. In turn, the plate 765 can be drawn generally along arrow C, thereby shortening a distance between the plate 765 and the proximal ends of the conductive segments 714. Additionally, or alternatively, a distance between the proximal end 715 of the segmented ablation electrode 712 and the distal end 716 of the segmented ablation electrode 712 can decrease. In these and other embodiments, the size of the oculus 718 at the proximal end 715 of the segmented ablation electrode 712 can decrease to the second size (e.g., the second diameter) shown in FIG. 7B. For the sake of comparison and clarity, a ghost profile 712′ of the ablation electrode 712 and a ghost profile 718′ of the oculus 718 (corresponding to the profile of the ablation electrode 712 and the profile of the oculus 718 as illustrated in FIG. 7A) are illustrated in FIG. 7B to better highlight a change in the size of the oculus 718 after twisting the plate 765. In other words, the size of the oculus 718 can be adjusted by twisting the plate 765. In some embodiments, the plate 765 can be twisted using finely threaded rod or screw (e.g., that extends the length of the catheter shaft between a handle and the plate 765), or using an external tool to adjust the oculus 718 of the segmented ablation electrode 712 prior to insertion of the tip section 701 into a patient.

In some embodiments, the conductive segments 714 can be independently or collectively energized (e.g., all at once or in subgroupings). For example, each of the conductive segments 714 can be electrically connected to a corresponding electrical lead either directly or via contact with one or more of the other conductive segments 714. As a specific example, each of the conductive segments 714 can be electrically connected directly to a unique electrical lead such that each of the conductive segments 714 can be independently energized. As another specific example, one of the conductive segments 714 can be electrically connected directly to an electrical lead and may be electrically connected to one or more of the other conductive segments 714 via contact. In this manner, the electrical lead can be used to energy the one and the one or more of the other conductive segments 714 as a group. Insulated regions can be used in some embodiments to electrically isolate one or more of the conductive segments 714 from one or more of the other conductive segments 714. In some embodiments, when a leaf 714 is energized, all points on the leaf 714 can be equipotential. In these and other embodiments, when the leaves 714 form the ablation electrode 712 and are energized, each leaf 714 can be equipotential with all the other leaves 714.

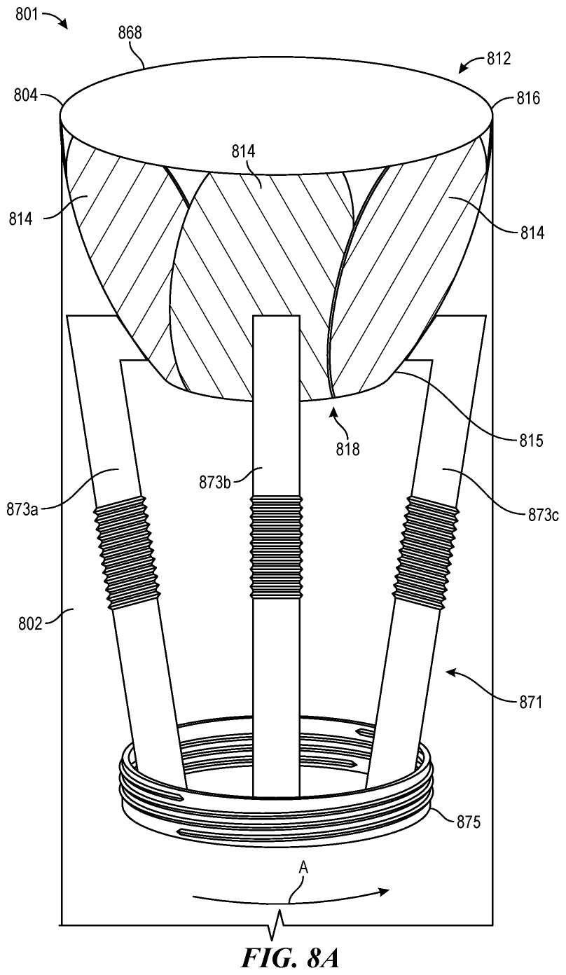

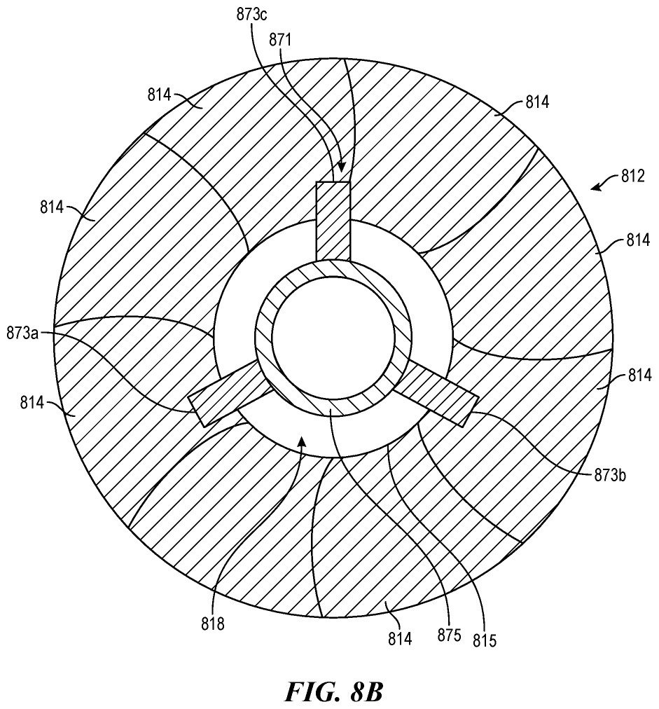

FIG. 8A is a partially schematic, transparent side view of another tip section 801 configured in accordance with various embodiments of the present technology, and FIG. 8B is a partially schematic rear view of the tip section 801 of FIG. 8A. The tip section 801 can be an example of the tip section 101 of FIG. 1, or another tip section of the present technology. As shown in FIG. 8A, the tip section 801 includes a catheter shaft 802 and a segmented ablation electrode 812 positioned within the catheter shaft 802. The segmented ablation electrode 812 includes a plurality of conductive segments 814. In the illustrated embodiment, the conductive segments 814 are leaves (e.g., sections of one or more segments) that are arranged in a partially overlapping manner to give the segmented ablation electrode 812 a generally dome shape or a generally partial dome shape. The segmented ablation electrode 812 is generally similar to the segmented ablation electrode 712 of FIGS. 7A and 7B. Therefore, similar reference numbers are used across FIGS. 7A-8B to denote identical or at least generally similar components.

In contrast with the tip section 701, the tip section 801 includes a collet 871 having a plurality of arms 873, three of which (arms 873a-873c) are shown in FIGS. 8A and 8B. The arms 873 of the collet 871 can be biased outward away from the segmented ablation electrode 812 but can be configured to apply an inward, squeezing force to a proximal end portion of the segmented ablation electrode 812. More specifically, the tip section 801 can include a ring or nut 875 about the arms 873 of the collet 871. In some embodiments, the nut 875 can be threaded and configured to engage with corresponding threading on the arms 873 of the collet 871. Thus, when twisted or rotated generally along or parallel to arrow A shown in FIG. 8A, the nut 875 can climb the arms 873 of the collet 871 toward the segmented ablation electrode 812 to thereby move distal ends of the arms 873 inward and apply a squeezing force to the segmented ablation electrode 812 to reduce the size of an oculus 818 at a proximal end 815 of the segmented ablation electrode 812. Twisting or rotating the 875 in a direction generally opposite to the arrow A can allow the nut 875 to descend along the arms 873 of the collet 871. In turn, the distal ends of the arms 873 can move outward, thereby allowing the proximal end portion of the segmented ablation electrode 812 to expand and increase the size of the oculus 818 at the proximal end 815 of the segmented ablation electrode 812. In other words, the size of the oculus 818 can be adjusted by twisting or rotating the nut 875. In some embodiments, the nut 875 can be twisted using finely threaded rod or screw (e.g., that extends the length of the catheter shaft between a handle and the nut 875), or using an external tool to adjust the oculus 818 of the segmented ablation electrode 812 (e.g., prior to insertion of the tip section 801 into a patient).

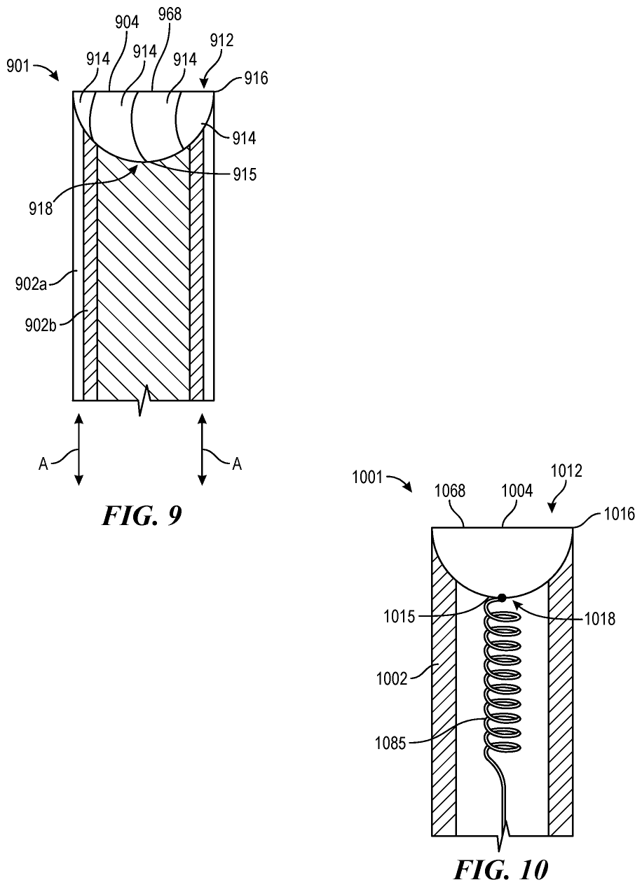

FIG. 9 is a partially schematic, transparent side view of another tip section 901 configured in accordance with various embodiments of the present technology. The tip section 901 can be an example of the tip section 101 of FIG. 1, or another tip section of the present technology. As shown, the tip section 901 includes a catheter shaft 902 having an outer first portion 902a and an inner second portion 902b, and a segmented ablation electrode 912 at least partially positioned within the catheter shaft 902. The segmented ablation electrode 912 includes a plurality of conductive segments 914. In the illustrated embodiment, the conductive segments 914 are leaves (e.g., sections of one or more segments) that are arranged in a partially overlapping manner to give the segmented ablation electrode 912 a generally dome shape or a generally partial dome shape. The segmented ablation electrode 912 is generally similar to the segmented ablation electrode 712 of FIGS. 7A and 7B and the segmented ablation electrode 812 of FIG. 8A and 8B. Therefore, similar reference numbers are used across FIGS. 7A-9 to denote identical or at least generally similar components.

In contrast with the other segmented ablation electrodes discussed in detail above, the segmented ablation electrode 912 of FIG. 9 is arranged such that it is at least partially positioned within the outer first portion 902a of the catheter shaft 902 but extends at least partially beyond the inner second portion 902b of the catheter shaft 902. More specifically, a proximal end portion of the segmented ablation electrode 912 can be seated within a distal end portion of the inner second portion 902b of the catheter shaft 902. In addition, the inner second portion 902b can be moveable generally along arrow A. Thus, as the inner second portion 902b is moved in a direction generally toward a distal end 916 of the segmented ablation electrode 912, the inner second portion 902b can apply a squeezing force to the proximal end portion of the segmented ablation electrode 912 and thereby decrease a size of an aperture or oculus 918 at a proximal end 915 of the segmented ablation electrode 912. As the inner second portion 902b is moved in a direction generally away from the distal end 916 of the segmented ablation electrode 912, the proximal end portion of the segmented ablation electrode 912 can be permitted to expand outward toward the outer first portion 902a and thereby increase a size of the oculus 918. In other words, the size of the oculus 918 can be adjusted by translating or moving the inner second portion 902b of the catheter shaft 902 generally along the arrow A.

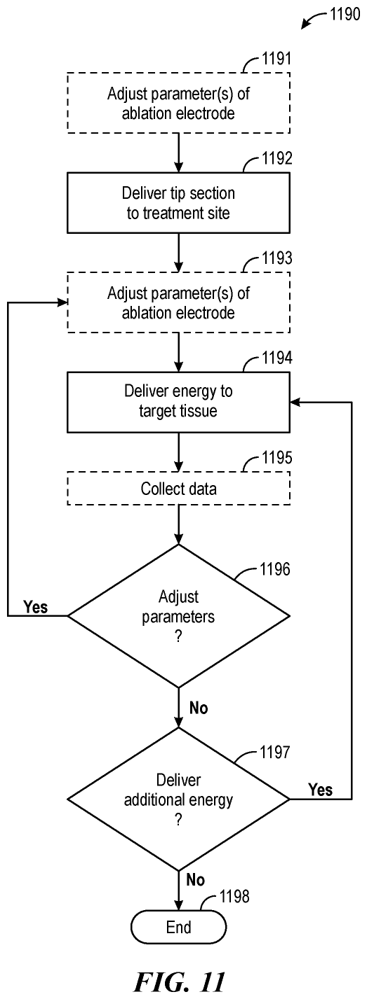

FIG. 10 is a partially schematic, transparent side view of another tip section 1001 configured in accordance with various embodiments of the present technology. The tip section 1001 can be an example of the tip section 101 of FIG. 1, or another tip section of the present technology. As shown, the tip section 1001 includes a catheter shaft 1002 and an ablation electrode 1012 at least partially positioned within the catheter shaft 1002. The ablation electrode 1012 can be segmented in some embodiments. For example, the ablation electrode 1012 can include a plurality of conductive segments (not shown), such as conductive leaves (e.g., sections of one or more segments) that are arranged in a partially overlapping manner to give the ablation electrode 1012 a generally dome shape or a generally partial dome shape. The ablation electrode 1012 can be generally similar to the segmented ablation electrode 712 of FIGS. 7A and 7B, the segmented ablation electrode 812 of FIG. 8A and 8B, and/or the segmented ablation electrode 912 of FIG. 9. Therefore, similar reference numbers are used across FIGS. 7A-10 to denote identical or at least generally similar components.

As shown, the tip section 1001 further includes a cord 1085 or other spring-like component. The cord 1085 can be attached to a proximal end 1015 of the 1012, and can be twisted (e.g., into the coiled configuration shown in FIG. 10). As the cord 1085 is twisted, the cord 1085 can pull proximal end portions of the conductive segments of the 1012 inward to reduce a size of an oculus 1018 at the proximal end 1015 of the electrode 1012. Additionally, or alternatively, the electrode 1012 can be movable relative to the 1002, such as along the major axis of the 1002. In these embodiments, as the cord 1085 is twisted, the electrode 1012 can be drawn proximally along the major axis of the 1002 and be collapsed inward to decrease the size of the oculus 1018. As the cord 1085 untwists or is otherwise relaxed, the proximal end portion of the electrode 1012 can be permitted to expand outward, thereby increasing the size of the oculus 1018 at the proximal end 1015 of the electrode 1012. In other words, the size of the oculus 1018 can be adjusted by twisting or untwisting the cord 1085.

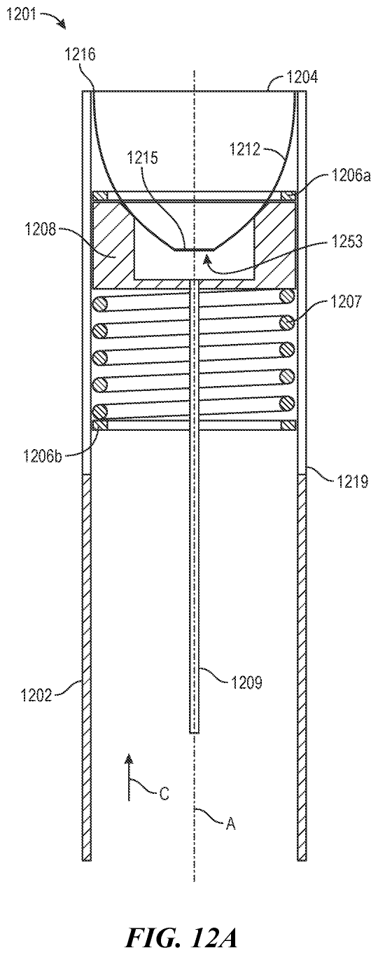

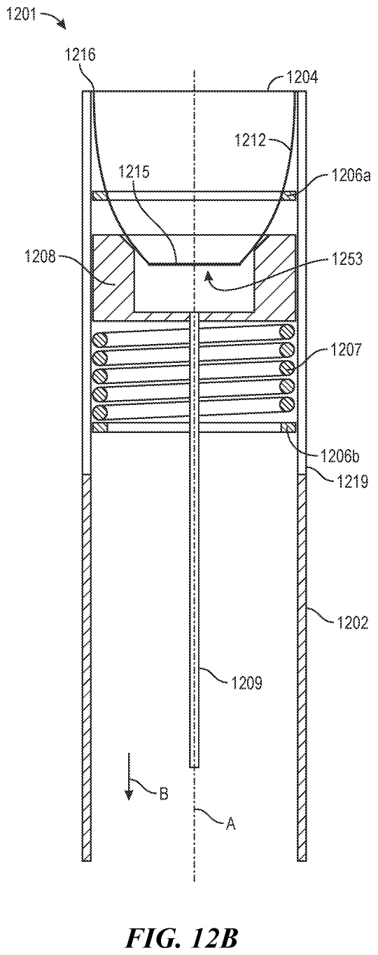

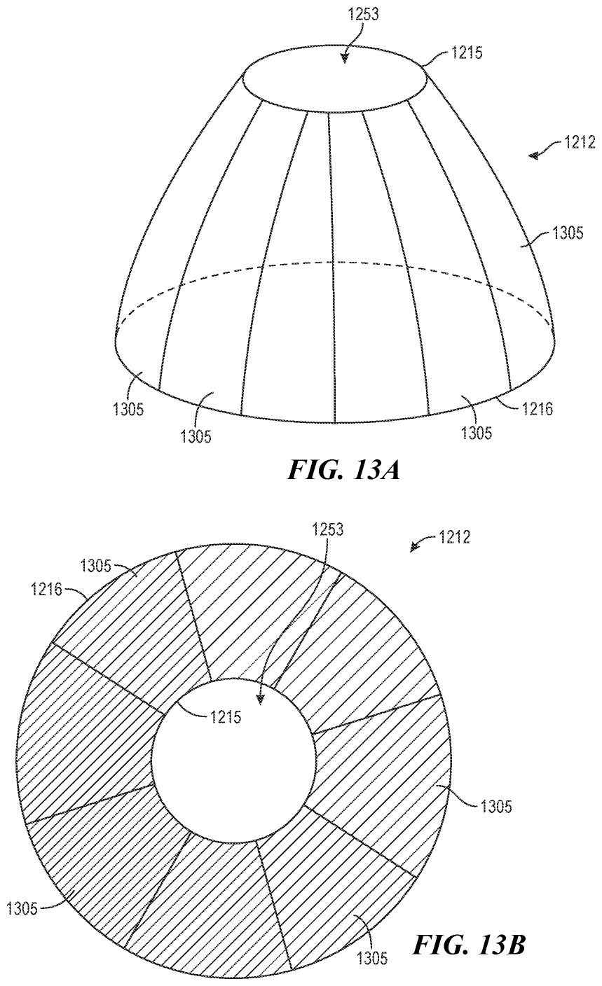

FIG. 11 is a flow diagram illustrating a method 1190 in accordance with various embodiments of the present technology. For example, the method 1190 can be a method of operation a catheter ablation system configured in accordance with various embodiments of the present technology. The method 1190 is illustrated as a series of steps or blocks. All or a subset of one or more of the blocks can be performed by a physician and/or by various components of a catheter ablation system (e.g., the catheter ablation system 100 of FIG. 1). In addition, all or a subset of one or more of the blocks can be performed in accordance with the disclosure provided above and/or with the discussion of FIGS. 12A-13B below.

The method 1190 optionally begins at block 1191 by adjusting one or more parameters related to an ablation electrode of a catheter ablation system. Adjusting the one or more parameters can include adjusting the one or more parameters using an external tool, using a mechanism/actuator (e.g., to adjust an amount of force proximally applied to/via a wire and/or spring), and/or prior to insertion of a tip section or another portion of a catheter of the catheter ablation system into a patient. Adjusting the one or more parameters can include adjusting an angle of a central axis of the ablation electrode, such as relative to a longitudinal axis of a shaft of the catheter and/or relative to an anticipated orientation of target tissue at a treatment site. Adjusting the one or more parameters can include adjusting one or more distances between conductive segments of the ablation electrode. In these and other embodiments, adjusting the one or more parameters can include adjusting an effective aperture/oculus or an effective size of an aperture/oculus of the ablation electrode. Additionally, or alternatively, adjusting the one or more parameters can include expanding or retracting the ablation electrode. In these and still other embodiments, adjusting the one or more parameters can include adjusting (e.g., modifying, selecting, tailoring, changing, altering) electrical connections between one or more conductive segments of the ablation electrode and one or more energy sources. Adjusting the one or more parameters can include selecting which of the conductive segments of the ablation electrode to energize to deliver ablative energy to target tissue at a treatment site. Adjusting the one or more parameters can include tailoring the one or more parameters such that the ablation electrode is configured to generate an electric field having a desired distribution or energy profile/pattern.

At block 1192, the method 1190 continues (or optionally begins) by delivering the tip section of the catheter to a treatment site. Delivering the tip section of the catheter to the treatment site can include delivering the tip section to a chamber of a heart of a patient (e.g., via a vein or artery of the patient, such as through the patient's groin). In these and other embodiments, delivering the tip section of the catheter to the treatment site can include delivering the tip section to a treatment site on or at a blood-tissue boundary, such as along an inner surface of a wall of the patient's heart.

At block 1193, the method 1190 optionally continues by adjusting one or more parameters related to an ablation electrode of the catheter ablation system. Adjusting the one or more parameters at block 1193 can be generally similar to adjusting one or more parameters at block 1191 above except that all or a subset of the parameters adjusted at block 1193 can be adjust in vivo while at least the tip section of the catheter is positioned within the patient. Additionally, or alternatively, adjusting the one or more parameters at block 1193 can include adjusting (e.g., changing) a grouping of which of the conductive segments are energized and/or are being used to deliver energy to tissue.

At block 1194, the method 1190 continues by delivering energy to target tissue. Delivering energy to target tissue can include energizing one or more conductive segments of the ablation electrode, such as using one or more energy sources. Delivering energy to target tissue can include modulating various parameters of electrical energy delivered to the one or more conductive segments. Delivering energy to target tissue can include generating, producing, emitting, or applying an electric field or an electric profile/pattern to target tissue. Delivering energy to target tissue can include ablating the target tissue.

At block 1195, the method 1190 optionally continues by collecting data related to the energy delivery at block 1194. Collecting the data can include collecting data corresponding to energy delivered to the ablation electrode and/or to the target tissue. For example, collecting the data can include collecting data corresponding to energy supplied to the ablation electrode from an energy source. Collecting the data can include collecting the data via sensors, circuit implementations (e.g., voltage detection circuits), or microcontrollers of the catheter ablation system. As another example, collecting the date can include collecting data corresponding to an energy profile or electric field applied or delivered to the target tissue via the ablation electrode.

At block 1196, the method 1190 continues by determining whether to adjust one or more parameters related to an ablation electrode of the catheter ablation system. Determining whether to adjust the one or more parameters can be based on data collected at block 1195 and/or on a predetermined schedule or routine of energy delivery. In the event the method 1190 determines to adjust the one or more parameters (block 1196: Yes), the method 1190 returns to block 1193. On the other hand, in the event the method 1190 determines not to adjust the one or more parameters (block 1196: No), the method 1190 proceeds to block 1197.

At block 1197, the method 1190 continues by determining whether to apply additional energy. Determining whether to apply additional energy can include determining whether to apply additional energy to the target tissue at the delivery site or to other target tissue at another delivery site. Additionally, or alternatively, determining whether to apply additional energy can based on data collected at block 1195 and/or on a predetermined schedule or routine of energy delivery. In the event the method 1190 determines to apply additional energy (block 1197: Yes), the method 1190 returns to block 1194. In the event the method 1190 determines to apply additional energy to other target tissue at another treatment/delivery site, the method 1190 can include repositioning the tip section of the catheter at the other treatment/delivery site before returning to block 1194. On the other hand, in the event the method 1190 determines not to apply additional energy (block 1197: No), the method 1190 terminates at block 1198.

FIGS. 12A and 12B are partially schematic, partially transparent side views of another tip section 1201 configured in accordance with various embodiments of the present technology. The tip section 1201 can be an example of the tip section 101 of FIG. 1, or another tip section configured in accordance with the present technology. As shown, the tip section 1201 includes a catheter shaft 1202 and an ablation electrode 1212 (e.g., a single-segmented ablation electrode) positioned within the catheter shaft 1202. In some embodiments, a distal end portion of the catheter shaft 1202 can be rigid and/or insulated. For example, the distal end portion of the catheter shaft 1202 can include a metal jacket 1219 to provide rigidity and/or insulation, and/or portions of the catheter shaft 1202 proximal its distal end portion can lack such a metal jacket (e.g., for flexibility).

FIGS. 13A and 13B are partially schematic views of the ablation electrode 1212 of FIGS. 12A and 12B. As shown, the ablation electrode 1212 includes a plurality of conductive leaves 1305 (e.g., a plurality of sections, each having one or more segments). The leaves 1305 can be flexible and/or clastic in some embodiments. In the illustrated embodiment, the conductive leaves 1305 are arranged in a partially overlapping manner to give the ablation electrode 1212 a generally dome shape or a generally partial dome shape. In some embodiments, when a leaf 1305 is energized, all points on the leaf can be equipotential. In these and other embodiments, when the leaves 1305 form the ablation electrode 1212 and are energized, each leaf 1305 can be equipotential with all the other leaves 1305.

Referring to FIGS. 12A-13B together, the leaves 1305 (FIGS. 13A and 13B) are shaped such that together they form the dome-shaped or partially-domed-shaped ablation electrode 1212 with an oculus 1253 at a proximal end 1215 of the ablation electrode 1212. Each of the conductive leaves 1305 can be attached to a rim 1204 (FIGS. 12A and 12B) at a distal end 1215 of the ablation electrode 1212. In some embodiments, when the ablation electrode 1212 formed by the leaves 1305 is not subjected to any forces from external sources, the ablation electrode 1212 can assume a shape having the oculus size shown in FIG. 12B.

As shown in FIGS. 12A and 12B, the tip section 1201 can include (a) two retaining rings 1206a and 1206b that are held at fixed locations within the tip section 1201, and (b) a cylinder 1208 that is positioned between the retaining rings 1206a and 1206b and that is configured to at least partially surround an exterior of the ablation electrode 1212 at or near its proximal end 1215. The tip section 1201 can further include a spring 1207 (e.g., a helical spring) that can be positioned between the retaining rings 1206a and 1206b, can be at least partially compressed, can abut the retaining ring 1206b at one end, and can exert a distal force on the cylinder 1208 at the other end. When the spring 1207 exerts an upwards force on the cylinder 1208, the cylinder 1208 can, in turn, exert a force on the ablation electrode 1212 such that the conductive leaves 1305 (FIGS. 13A and 13B) move closer to each other at their proximal ends, thereby reducing the size of the oculus of the ablation electrode 1212 toward the oculus size shown in FIG. 12A. When the cylinder 1208 is advanced distally (e.g., via the upwards force from the spring 1207) to a point where its distal end abuts the distal retaining ring 1206a, the retaining ring 1206a can prevent further distal movement of the cylinder 1208.

A proximal end of the cylinder 1208 can be bar-shaped such that the cylinder (i) is not completely or fully closed off at its proximal end and (ii) permits fluid to flow through the cylinder 1208. A pull wire 1209 can be attached to the bar-shaped proximal end of the cylinder 1208 and extend within the catheter shaft 1202 toward a proximal end of the catheter shaft 1202 to a mechanism/actuator (not shown) that allows an operator to apply pulling forces on the cylinder 1208 via the wire 1209 and generally along an axis A in the general direction of arrow B (FIG. 12B). When the wire 1209 is pulled in the general direction of arrow B, the cylinder 1208 can be pulled proximally and compress (e.g., further compress) the spring 1207. In turn, the force exerted by the cylinder 1208 on the exterior of the ablation electrode 1212 can be reduced and/or the location along an exterior of the ablation electrode 1212 at which the cylinder 1208 exerts the force can shift proximally, allowing the conductive leaves 1305 (FIGS. 13A and 13B) of the ablation electrode 1212 to move (e.g., straighten) in a manner that increases the size (e.g., diameter) of the oculus at the proximal end of the ablation electrode 1212 (e.g., toward the state of the ablation electrode 1212 shown in FIG. 12B). As pulling forces on the wire 1209 in the general direction of arrow B are decreased, the upward force exerted on the cylinder 1208 by the spring 1207 can move the cylinder 1208 distally along the axis A and in the general direction of arrow C (FIG. 12A). In turn, the force exerted by the cylinder 1208 on the exterior of the ablation electrode 1212 can be increased and/or the location along the exterior of the ablation electrode 1212 at which the cylinder 1208 exerts the force can shift distally, causing the conductive leaves 1305 of the ablation electrode 1212 to move (e.g., bend) in a manner that decreases the size (e.g., diameter) of the oculus at the proximal end of the ablation electrode (e.g., toward the state of the ablation electrode 1212 shown in FIG. 12A)

Although the segmented ablation electrode 212, the segmented ablation electrode 612, the segmented ablation electrode 712, the segmented ablation electrode 812, the segmented ablation electrode 912, the electrode 1012, and the ablation electrode 1212 are described in detail above as being formed of a plurality of conductive segments or leaves, other arrangements, configurations, and/or formations of ablation electrodes are of course possible and within the scope of the present technology. For example, an ablation electrode of the present technology can be formed of a (e.g., single, continuous, uniform) sheet of a conductive, pliable material that is corrugated/scored and/or can be folded into an arrangement similar to the segmented ablation electrodes 712, 812, 912, and/or 1012 illustrated in FIGS. 7A-10 above and/or to the ablation electrode 1212 illustrated in FIG. 12 above.

### C. Conclusion

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order above, alternative embodiments may perform steps in a different order. Furthermore, the various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. To the extent any material incorporated herein by reference conflicts with the present disclosure, the present disclosure controls.

Where the context permits, singular or plural terms may also include the plural or singular term, respectively. In addition, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Furthermore, as used herein, the phrase “and/or” as in “A and/or B” refers to A alone, B alone, and both A and B. Additionally, the terms “comprising,” “including,” “having,” and “with” are used throughout to mean including at least the recited feature(s) such that any greater number of the same features and/or additional types of other features are not precluded. Moreover, as used herein, the phrases “based on,” “depends on,” “as a result of,” and “in response to” shall not be construed as a reference to a closed set of conditions. For example, an exemplary step that is described as “based on condition A” may be based on both condition A and condition B without departing from the scope of the present disclosure. In other words, as used herein, the phrase “based on” shall be construed in the same manner as the phrase “based at least in part on” or the phrase “based at least partially on.”

From the foregoing, it will also be appreciated that various modifications may be made without deviating from the disclosure or the technology. For example, one of ordinary skill in the art will understand that various components of the technology can be further divided into subcomponents, or that various components and functions of the technology may be combined and integrated. In addition, certain aspects of the technology described in the context of particular embodiments may also be combined or eliminated in other embodiments. Furthermore, although advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. An ablation catheter, comprising:
a shaft having a proximal end and a distal end opposite the proximal end; and
an ablation electrode at the distal end of the shaft, the ablation electrode including a first conductive segment and a second conductive segment different from the first conductive segment,
wherein the first conductive segment and the second conductive segment are arranged in a stack along a common axis, and
wherein the first conductive segment and the second conductive segment are independently energizable.

2. The ablation catheter of claim 1 wherein:
the first conductive segment is a first conductive ring; and
the second conductive segment is a second conductive ring.

3. The ablation catheter of claim 1 wherein the first conductive segments and the second conductive segment are concentrically arranged in the stack.

4. The ablation catheter of claim 1 wherein:
the first conductive segment is a first conductive ring and the second conductive segment is a second conductive ring;
the first conductive ring has a first radius; and
the second conductive ring has a second radius different from the first radius.

5. The ablation catheter of claim 1 wherein:
the first conductive segment and the second conductive segment are arranged in the stack such that the ablation electrode has a partial dome shape with a central axis;
the ablation electrode has a proximal end and a distal end opposite the proximal end; and
radii of cross-sections of the ablation electrode along planes perpendicular to the central axis increase from the proximal end of the ablation electrode to the distal end of the ablation electrode.

6. The ablation catheter of claim 5 wherein:
the shaft includes a longitudinal axis; and
the ablation electrode is positioned at the distal end of the shaft such that the central axis of the partial dome shape is arranged generally perpendicular to the longitudinal axis of the shaft.

7. The ablation catheter of claim 5 wherein:
the shaft includes a longitudinal axis; and
the ablation electrode is positioned at the distal end of the shaft such that the central axis of the partial dome shape is arranged generally parallel to the longitudinal axis of the shaft.

8. (canceled)

9. (canceled)

10. The ablation catheter of claim 1 wherein the ablation electrode includes an oculus at a proximal end portion of the ablation electrode.

11. The ablation catheter of claim 10 wherein an effective diameter of the oculus is adjustable.

12. The ablation catheter of claim 10 wherein the oculus is centered on a central axis of the ablation electrode.

13. The ablation catheter of claim 10 wherein the oculus is off-centered from a central axis of the ablation electrode.

14. The ablation catheter of claim 1 wherein:
the first conductive segment at least partially defines a first aperture having a first diameter;
the second conductive segment at least partially defines a second aperture having a second diameter different from the first diameter; and
an effective oculus of the ablation electrode is adjustable between the first aperture and the second aperture.

15. The ablation catheter of claim 1, further comprising a second ablation electrode at a location along the shaft, the second ablation electrode including a third conductive segment and a fourth conductive segment different from the third conductive segment.

16. The ablation catheter of claim 15 wherein:
the ablation electrode includes a first central axis;
the second ablation electrode includes a second central axis; and
the ablation electrode and the second ablation electrode are positioned such that the first central axis and the second central axis are arranged parallel with one another.

17. The ablation catheter of claim 15 wherein:
the ablation electrode includes a first central axis;
the second ablation electrode includes a second central axis; and
the ablation electrode and the second ablation electrode are positioned such that the first central axis and the second central axis are arranged non-parallel with one another.

18-20. (canceled)

21. The ablation catheter of claim 1 wherein the first conductive segment is physically separated from the second conductive segment via an insulated region positioned between the first conductive segment and the second conductive segment.

22. The ablation catheter of claim 1 wherein the first conductive segment and the second conductive segment are configurable such that the first conductive segment and the second conductive segment are simultaneously energizable.

23. The ablation catheter of claim 1 wherein:
the first conductive segment, when energized, has a first energy profile; and
the second conductive segment, when energized, has a second energy profile different from the first energy profile.

24. (canceled)

25. The ablation catheter of claim 1 wherein the second conductive segment is generally cylindrical and is positioned distal the first conductive segment.

26. The ablation catheter of claim 1 wherein the first conductive segment is a conic section.

27-71. (canceled)
